(19) United States
(12) Patent Application Publication
(10) Pub. No.: US 2009/0227518 A1
(43) Pub. Date: Sep. 10, 2009

(54) COMPOSITIONS AND METHODS FOR TREATING ACTIN-MEDIATED MEDICAL CONDITIONS

(75) Inventors: Leland Shapiro, Denver, CO (US); Gregory Pott, Westminster, CO (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(73) Assignee: The Regents of the University of Colorado, Boulder, CO (US)

(21) Appl. No.: 11/719,200

(22) PCT Filed: Dec. 4, 2006
(86) PCT No.: PCT/US2006/061577

§ 371 Date: May 11, 2007

Related U.S. Application Data
(60) Provisional application No. 60/742,191, filed on Dec. 2, 2005.

(51) Int. Cl. A61K 38/08

(52) U.S. Cl. 514/17

(57) ABSTRACT

The present invention provides a method for treating a subject an actin-mediated medical condition by administering to the subject an actin modulator. In some embodiments, the actin-mediated medical condition is an intracellular actin-mediated medical condition. In other embodiments, the actin-mediated medical condition is a cellular-surface actin mediated medical condition.

### CROSS-REFERENCE TO RELATED APPLICATIONS

This application claims the priority benefit of U.S. Provisional Application No. 60/742,191, filed Dec. 2, 2005, which is incorporated herein by reference in its entirety. Filing of this Application on Dec. 4, 2006, is timely because Dec. 2, 2006, is a Saturday.

### FIELD OF THE INVENTION

The present invention relates to methods for treating actin-mediated medical conditions.

### BACKGROUND OF THE INVENTION

Actin is a ubiquitous globular structural protein that polymerizes in a helical fashion to form an actin filament (or microfilament). These filaments form the cytoskeleton—a three-dimensional network inside a eukaryotic cell. Actin filaments provide mechanical support for the cell, determine the cell shape and participate in certain cell junctions, in cytoplasmic streaming and in contraction of the cell during cytokinesis. In the cytosol, actin is predominantly bound to ATP, but can also bind to ADP. An ATP-actin complex polymerizes faster and dissociates slower than an ADP-actin complex. Actin is one of the most highly conserved proteins.

The cytoskeleton is like a cellular scaffolding or skeleton contained, as all other organelles, within the cytoplasm. It is contained in all eukaryotic cells. It is a dynamic structure that maintains cell shape, and also has been known to protect the cell, and plays important roles in both intra-cellular transport (the movement of vesicles and organelles, for example) and cellular division.

Within epithelial cells, filamentous actin is concentrated at the plasma membrane where a wide variety of actin-associated proteins harness the potential and structure of actin filaments to moderate functions at the plasma membrane. These functions include structural support of the plasma membrane, establishing and maintaining cell polarity, regulation of membrane protein distribution and activity and enhancing membrane vesicle trafficking. Consequently, the actin cytoskeleton contributes significantly to the cellular pathogenesis in a number of disease states.

In some instances, defect in actin or its associated proteins results in neutrophil cytoskeletal disease where abnormality primarily appears as motility or chemotactic defect of the cells. Neutrophils and other phagocytes play an important role in immune system. These cells migrate to the site of infection, ingest pathogens, and destroy them after releasing granule contents and active oxygen. These activities of the cells are associated with a reorganization of the cytoskeleton where actin polymerizes, cross-links, anchors to the membrane and depolymerizes under the control of various actin-associated proteins.

Because of the many functions of actin in the onset and progression of health conditions, there is a need to modulate actin activities to attenuate or inhibit the onset and/or progression of actin-mediated medical conditions.

### SUMMARY OF THE INVENTION

One aspect of the invention provides a method for treating a subject for actin-mediated medical conditions. Methods of the invention comprise administering to the subject a therapeutically effective amount of an actin inhibitor, whereby the medical condition of the subject is treated at least in part due to modulation of the cell-associated actin activity by the substance.

In some aspects of the invention, the actin-mediated medical condition is an intracellular actin-mediated medical condition.

In some embodiments, the actin modulator has no significant serine protease activity.

Methods of the invention are applicable in treating any medical conditions that involve cell-associated actin activity. In some embodiments of the invention, the medical condition is selected from the group consisting of viral infection, bacterial infection, conditions mediated by pro-inflammatory cytokine production, conditions mediated by nitric oxide (NO) production, a tumor, and graft rejection. Within these embodiments, in some instances the medical condition is mediated by bacteria-produced toxins. In other instances, the medical condition is a retroviral infection, in certain cases the medical condition is human immunodeficiency virus (HIV) infection. Still in other instances, the medical condition is infection and/or disease due to herpes simplex viruses, cytomegalovirus, influenza viruses, or varila (smallpox) virus. Yet in some instances, the medical condition is mycobacterial infection.

In some embodiments, the medical condition is a disease mediated by apoptosis or NO production. Within these embodiments, in some instances the apoptosis mediated disease is selected from the group consisting of sepsis, bacterial meningitis, and ischemia-reperfusion injury. In some cases, ischemia-reperfusion injury is selected from the group consisting of myocardial infarction, stroke, ischemic nephropathy, acute respiratory distress syndrome (ARDS), and shock liver.

Yet in other embodiments of the invention, the medical condition is selected from the group consisting of lung transplant, liver transplant, heart transplant, kidney transplant, bone marrow transplant, appendage transplant, face transplant, and pancreatic islet transplant.

A variety of cancers can be treated using methods of the invention. In one particular embodiment, methods of the invention are used to treat a cancer selected from the group consisting of fibrosarcoma, myxosarcoma, liposarcoma, chondrosarcoma, osteogenic sarcoma, chordoma, angiosarcoma, endotheliosarcoma, lymphangiosarcoma, Kaposi's sarcoma, lymphangioendotheliosarcoma, synovioma, mesothelioma, Ewing's tumor, leiomyosarcoma, rhabdomyosarcoma, rhabdosarcoma, colorectal carcinoma, pancreatic cancer, breast cancer, ovarian cancer, prostate cancer, melanoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, myeloma, lymphoma, and leukemia.

Methods of the invention are also useful in treating medical conditions involving pro-inflammatory cytokines or NO production. Accordingly, in some embodiments methods of the invention are used to treat a medical condition is selected from the group consisting of systemic lupus erythematosis (SLE, or lupus), rheumatoid arthritis, inflammatory bowel disease, sepsis, and sero-negative spondyloarthropathies.

Generally, any substance that is capable of modulating cell-associated actin activity can be used in methods of the invention. While substances that are useful in methods of the invention can have other biological activity, it should be appreciated that the medical condition of the subject is treated at least in part due to modulation of the cell-associated actin activity by the substance. Accordingly, as stated above medical conditions to be treated using methods of the invention involve conditions that are due to cellular-actin activity.

In some embodiments, the actin modulator is an actin-binding molecule or an antagonist of actin polymerization.

Still in other embodiments, the actin modulator inhibits the polymerization of actin.

In one particular embodiment, the actin modulator is selected from the group consisting of Gelsolin, latrunculin, vitrunculin, cytochalasin D, anti-actin antibodies, or a derivative or an analog thereof.

In other embodiments, the actin modulator is α₁-antitrypsin (AAT) or a member of the SERPIN (serine protease inhibitor) family of natural proteins, or a derivative thereof. Within these embodiments, in some instances the actin modulator is α₁-antitrypsin (AAT) or a derivative thereof. Within these instances, in some cases the actin modulator is selected from the group consisting of FVFLM (SEQ ID NO: 1), FVFAM (SEQ ID NO:2), FVALM (SEQ ID NO:3), FVFLA (SEQ ID NO:4), FLVFI (SEQ ID NO:5), FLMII (SEQ ID NO:6), FLFVL (SEQ ID NO:7), FLFVV (SEQ ID NO:8), FLFLI (SEQ ID NO:9), FLFFI (SEQ ID NO:10), FLMFI (SEQ ID NO:11), FMLLI (SEQ ID NO:12), FIIMI (SEQ ID NO:13), FLFCI (SEQ ID NO:14), FLFAV (SEQ ID NO:15), FVYLI (SEQ ID NO:16), FAFLM (SEQ ID NO:17), AVFLM (SEQ ID NO:18), and a combination thereof.

Another aspect of the invention provides a method for treating a subject for actin-mediated medical condition, said method comprising administering to the subject a therapeutically effective amount of an actin modulator that binds to cell-surface actin, whereby the medical condition of the subject is treated at least in part due to modulation of the cell-surface actin activity by the actin modulator, e.g., by binding to the cell-surface actin and altering intracellular actin structure.

### BRIEF DESCRIPTION OF THE DRAWINGS

FIG. 1 represents an example of an intracellular actin modulator affect on HIV.

FIG. 2 represents an example of an intracellular actin modulator affect on blood IL-6 in the blood.

FIG. 3 represents a schematic of an intracellular actin modulator interaction with actin.

FIG. 4 represents an exemplary electrophoresis gel of interaction of an intracellular actin modulator with a macromolecule.

FIG. 5 represents an example of an intracellular actin modulator affect on actin polymerization in a cell free system.

FIG. 6 represents an example of an intracellular actin modulator to bind to the surface of a cell in the presence or absence of a cytokine.

FIG. 7 represents an example of an intracellular actin modulator to bind to the surface of stimulated and unstimulated cells.

FIG. 8 represents an example of the fate of an intracellular actin modulator bound to the surface of a cell.

FIG. 9A-9C represents an example of an intracellular actin modulator binding to different forms of a cytoskeletal protein.

FIGS. 10A and 10B represents exemplary electrophoretic separation of an intracellular actin modulator to bind to a cytoskeletal protein at different ratios.

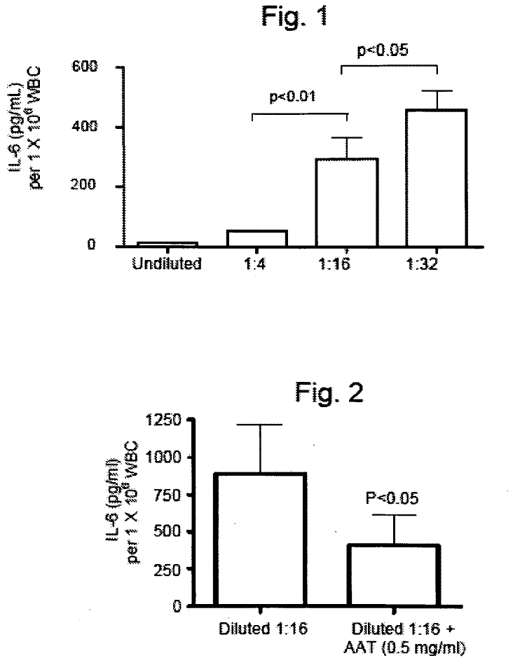

FIGS. 1A and 11B represents an exemplary electrophoretic gel (A) and a histogram representation of an intracellular actin modulator alone or in the presence of actin.

FIG. 12 represents a histogram of the data of Table 5.

### DETAILED DESCRIPTION OF THE INVENTION

A therapeutically effective amount means the amount of a compound that, when administered to a mammal for treating a disease, is sufficient to effect such treatment for the disease. The therapeutically effective amount will vary depending on the compound, the disease and its severity and the age, weight, etc., of the mammal to be treated.

Treating or treatment of a disease includes: (1) preventing the disease, i.e., causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

Modulation refers to a change in the level or magnitude of an activity or process. The change can be either an increase or a decrease. Modulation can be assayed by determining any parameter that directly or indirectly quantifies the protein or affects the protein activity.

Cancer, tumor and other similar terms refer to any neoplasm whether benign or malignant, and regardless of whether it has metastisized from the location of the cancer or tumor.

Medical condition refers to a condition in a subject due to a particular condition, e.g., actin stimulation. It should be appreciated that medical condition refers to a particular state of the subject regardless of whether the subject manifests any symptoms.

One aspect of the invention provides a method for treating actin-mediated medical condition. In some embodiments, the medical condition is at least in part the result of modulation of an intracellular actin. Still in other embodiments, the medical condition is at least in part the result of modulation of a cellular-surface actin.

It should be appreciated that methods of the invention is at least due in part to modulation of the actin activity by the actin modulator. Actin modulator can prohibit polymerization of actin or can be an actin-binding molecule.

In some embodiments, α₁-antitrypsin (AAT) protein, portions of the AAT sequence, homologs, analogs, derivatives, and peptide mimetics of part or all of the AAT sequence can be used to treat the medical condition.

In other embodiments, known inhibitors of actin including, but not limited to, Gelsolin, latrunculin, vitrunculin, cytochalasin D and anti-actin antibodies, antibody fragments or antibody analogs, or a combination thereof can be used. In certain instances, methods of the invention excludes using AAT or derivatives thereof.

Diseases or conditions of interest include, but are not limited to, viral infections, including retroviral infection such as human immunodeficiency virus (HIV-1) infection, bacterial infections including mycobacterial infections, anthrax-associated disease, nitric oxide (NO) mediated conditions, diseases characterized by increased pro-inflammatory cytokine production, diseases mediated by apoptosis, and graft rejection.

While AAT is currently known for its serine protease inhibition activity, surprising and unexpectedly the present inventors have found that AAT also modulates actin activity. Accordingly, it has been found that medical conditions mediated by actin activity, e.g., polymerization, can be treated by administering AAT or other actin modulators. Often actin modulators that are useful in methods of the invention inhibit or reduce actin activity.

AAT is a glycoprotein of MW 51,000 with 394 amino acids and 3 oligosaccharide side chains. Human AAT is a single polypeptide chain with no internal disulfide bonds and only a single cysteine residue normally intermolecularly disulfide-linked to either cysteine or glutathione. The reactive site at position 358 of AAT contains a methionine residue, which is labile to oxidation upon exposure to tobacco smoke or other oxidizing pollutants. Such oxidation may reduce the serine protease inhibitor activity of AAT; therefore substitution of another amino acid at that position, i.e., alanine, valine, glycine, phenylalanine, arginine or lysine, produces a form of AAT which is more stable as an inhibitor of serine proteases. AAT can be represented by SEQ ID NO:61. However, as stated above, in some embodiments of the invention the actin modulator has reduced or substantially no serine protease inhibitor activity.

SEQ ID NO:61

MPSSVSWGILLLAGLCCLVPVSLAEDPQGDAAQKTDTSHHDQDHPTFNKI
TPNLAEFAFSLYRQLASTNIEFSPVSIATAFAMLSLGTKADTHDEILEGL
NFNLTEIPEAQIHEGFQELLRTLNQPDSQLQLTTGNGLFLSEGLKLVDKF
LEDVKKLYHSEAFTVNFGDTEEAKKQINDYVEKGTQGKIVDLVKELDRDT
VFALVNYIFFKGKWERPFEVKDTEEEDFHVDQVTTVKVPMMKRLGMFNIQ
HCKKLSSWVLLMKYLGNATAIFFLPDEGKLQHLENELTHDIITKFLENED
RRSASLHLPKLSITGTYDLKSVLGQLGITKVFSNGADLSGVTEEAPLKLS
KAVHKAVLTIDEKGTEAAGAMFLEAIPMSIPPEVKFNKPFVFLMIEQNTK
SPLFMGKVVNPTQK

Details of the AAT sequence can be found, for example, in U.S. Pat. No. 5,470,970, incorporated herein by reference in its entirety.

Also contemplated is a series of peptides comprising carboxy terminal amino acid peptides corresponding to part of the AAT sequence. Among this series of peptides, those of interest include, but are not limited to, the following:

| | |
|---|---|
| FVFLM; | (SEQ ID NO:1) |
| FVFAM; | (SEQ ID NO:2) |
| FVALM; | (SEQ ID NO:3) |
| FVFLA; | (SEQ ID NO:4) |
| FLVFI; | (SEQ ID NO:5) |
| FLMII; | (SEQ ID NO:6) |
| FLFVL; | (SEQ ID NO:7) |
| FLFVV; | (SEQ ID NO:8) |
| FLFLI; | (SEQ ID NO:9) |
| FLFFI; | (SEQ ID NO:10) |
| FLMFI; | (SEQ ID NO:11) |
| FMLLI; | (SEQ ID NO:12) |
| FIIMI; | (SEQ ID NO:13) |
| FLFCI; | (SEQ ID NO:14) |
| FLFAV; | (SEQ ID NO:15) |
| FVYLI; | (SEQ ID NO:16) |
| FAFLM; | (SEQ ID NO:17) |
| AVFLM; | (SEQ ID NO:18) |

and a combination thereof.

The actin modulator can be a small molecule or a peptide. The peptides can be homologous and analogous of peptides. Peptide homologues are natural peptides with sequence homology, and peptide analogues are peptidyl derivatives, e.g., aldehyde or ketone derivatives of such peptides. Typical examples of analogues are oxadiazole, thiadiazole and triazole peptoids. Without limiting to AAT and peptide derivatives of AAT, in certain embodiments actin modulators are oxadiazole, thiadiazole and triazole peptoids.

In other embodiments, the actin modulator is an amino acid peptide corresponding to 10 amino acid fragments of AAT including, but not limited to, the following amino acid peptides:

| | |
|---|---|
| MPSSVSWGIL; | (SEQ ID NO:19) |
| LAGLCCLVPV; | (SEQ ID NO:20) |
| SLAEDPQGDA; | (SEQ ID NO:21) |
| AQKTDTSHHD; | (SEQ ID NO:22) |
| QDHPTFNKIT; | (SEQ ID NO:23) |
| PNLAEFAFSL; | (SEQ ID NO:24) |
| YRQLAHQSNS; | (SEQ ID NO:25) |
| TNIFFSPVSI; | (SEQ ID NO:26) |
| ATAFAMLSLG; | (SEQ ID NO:27) |
| TKADTHDEIL; | (SEQ ID NO:28) |
| EGLNFNLTEI; | (SEQ ID NO:29) |
| PEAQIHEGFQ; | (SEQ ID NO:30) |
| ELLRTLNQPD; | (SEQ ID NO:31) |
| SQLQLTTGNG; | (SEQ ID NO:32) |
| LFLSEGLKLV; | (SEQ ID NO:33) |
| DKFLEDVKKL; | (SEQ ID NO:34) |
| YHSEAFTVNF; | (SEQ ID NO:35) |
| GDHEEAKKQI; | (SEQ ID NO:36) |
| NDYVEKGTQG; | (SEQ ID NO:37) |
| KIVDLVKELD; | (SEQ ID NO:38) |
| RDTVFALVNY; | (SEQ ID NO:39) |
| IFFKGKWERP; | (SEQ ID NO:40) |
| FEVKDTEDED; | (SEQ ID NO:41) |
| FHVDQVTTVK; | (SEQ ID NO:42) |
| VPMMKRLGMF; | (SEQ ID NO:43) |
| NIQHCKKLSS; | (SEQ ID NO:44) |
| WVLLMKYLGN; | (SEQ ID NO:45) |
| ATAIFFLPDE; | (SEQ ID NO:46) |
| GKLQHLENEL; | (SEQ ID NO:47) |
| THDIITKFLE; | (SEQ ID NO:48) |
| NEDRRSASLH; | (SEQ ID NO:49) |
| LPKLSITGTY; | (SEQ ID NO:50) |
| DLKSVLGQLG; | (SEQ ID NO:51) |
| ITKVFSNGAD; | (SEQ ID NO:52) |
| LSGVTEEAPL; | (SEQ ID NO:53) |
| KISKAVHKAV; | (SEQ ID NO:54) |
| LTIDEKGTEA; | (SEQ ID NO:55) |
| AGAMFLEAIP; | (SEQ ID NO:56) |
| MSIPPEVKFN; | (SEQ ID NO:57) |
| KPFVFLMIEQ; | (SEQ ID NO:58) |
| NTKSPLFMGK; | (SEQ ID NO:59) |
| VVNPTQK; | (SEQ ID NO:60) |

or any combination thereof.

In some embodiments, AAT or its homologs or analogs are dosed to a subject in the range from about 10 ng to about 10 mg per mL of biologic fluid of treated patient. The therapeutically effective amount of AAT peptides or other actin modulator can also range from about 1 nM to about 1 mM in biologic fluid of a treated patient.

### Pharmaceutical Composition

The actin modulators of the present invention can be administered to a patient to achieve a desired physiological effect. Typically the patient is an animal, more often a mammal, and most often a human. The actin modulator can be administered in a variety of forms adapted to the chosen route of administration, i.e., orally or parenterally. Parenteral administration in this respect includes administration by the following routes: intravenous; intramuscular; subcutaneous; intraocular; intrasynovial; transepithelially including transdermal, ophthalmic, sublingual and buccal; topically including ophthalmic, dermal, ocular, rectal and nasal inhalation via insufflation and aerosol; intraperitoneal; and rectal systemic.

The actin modulator can be orally administered, for example, with an inert diluent or with an assimilable edible carrier, or it can be enclosed in hard or soft shell gelatin capsules, or it can be compressed into tablets, or it can be incorporated directly with the food of the diet. For oral therapeutic administration, the actin modulator can be incorporated with excipient and used in the form of ingestible tablets, buccal tablets, troches, capsules, elixirs, suspensions, syrups, wafers, and the like. Such compositions and preparation can contain at least 0.1% of actin modulator. The percentage of the compositions and preparation can, of course, be varied and can conveniently be between about 1 to about 10% of the weight of the unit. The amount of actin modulator in such therapeutically useful compositions is such that a suitable dosage will be obtained. Typical compositions or preparations according to the invention are prepared such that an oral dosage unit form contains from about 1 to about 1000 mg of actin modulator.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the actin modulator, sucrose as a sweetening agent, methyl and propylparabens a preservatives, a dye and flavoring such as cherry or orange flavor. Of course, any material used in preparing any dosage unit form should be pharmaceutically pure and substantially non-toxic in the amounts employed. In addition, the actin modulator can be incorporated into sustained-release preparations and formulation.

The actin modulator can also be administered parenterally. Solutions of the actin modulator as a free base or pharmacologically acceptable salt can be prepared in water suitably mixed with a surfactant such as hydroxypropylcellulose. Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacterial and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, e.g., sugars or sodium chloride. Prolonged absorption of the injectable compositions of agents delaying absorption, e.g., aluminum monostearate and gelatin.

Sterile injectable solutions are prepared by incorporating the actin modulator in the required amount in the appropriate solvent with various other ingredients enumerated above, as required, followed by filtered sterilization. Generally, dispersions are prepared by incorporating the various sterilized active ingredient into a sterile vehicle which contains the basic dispersion medium and the required other ingredients from those listed above. In the case of sterile powders for the preparation of sterile injectable solutions, the typical methods of preparation are vacuum drying and the freeze drying technique which yield a powder of the active ingredient plus any additional desired ingredient from previously sterile-filtered solution thereof.

The actin modulators of the invention can be administered to a mammal alone or in combination with pharmaceutically acceptable carriers, as noted above, the proportion of which is determined by the solubility and chemical nature of the actin modulator, chosen route of administration and standard pharmaceutical practice.

The physician will determine, the dosage of the present therapeutic agents which will be most suitable for treatment and it will vary with the form of administration and the particular actin modulator chosen, and also, it will vary with the particular patient under treatment. The physician will generally wish to initiate treatment with small dosages by small increments until the optimum effect under the circumstances is reached. The therapeutic dosage can generally be from about 0.1 to about 6000 mg/day, and preferably from about 10 to about 1000 mg/day, or from about 0.1 to about 50 mg/Kg of body weight per day and preferably from about 0.1 to about 20 mg/Kg of body weight per day and can be administered in several different dosage units. Higher dosages, on the order of about 2× to about 4×, may be required for oral administration.

Additional objects, advantages, and novel features of this invention will become apparent to those skilled in the art upon examination of the following examples thereof, which are not intended to be limiting.

### EXAMPLES

### AAT Suppression of HIV-1 Replication

HIV-1 replicates in vitro following exposure to several pro-inflammatory cytokines. Examples of HIV-1 replication-inducing cytokines include interleukin (IL)-1, IL-6, and tumor necrosis factor (TNF)α. The HIV-1-inducing effect of these pro-inflammatory cytokines suggests a link between HIV-1 infection and inflammation. Several publications report that HIV-1 is induced to replicate substantially in HIV-1-infected persons who are exposed to pro-inflammatory insults like co-infection with a separate agent (e.g., bacteria that cause pneumonia), or following immunization with influenza vaccine or tetanus vaccine. An antiretroviral effect for AAT in human whole blood was demonstrated and one potential mechanism by which AAT produced this effect was by suppressing HIV-1-inducing pro-inflammatory cytokines. Studies have shown that reducing AAT concentrations by small amounts markedly reduced its antiretroviral effect. If AAT is an anticytokine agent as well as an antiretroviral agent in whole blood, then dilution of whole blood would reduce the AAT concentration (and hence reduce its antiretroviral effect) and increase cytokine production.

### General Procedure and Materials

An intracellular actin binding molecule, α₁-antitrypsin (AAT) used in these studies was purified from the blood of healthy volunteers. Preparations of AAT used in these experiments were obtained from both the Red Cross and Baxter (Aralast™, Westlake Village, Calif.). AAT was purified to single-band homogeneity. The Red Cross AAT protein was diafiltered into a diluent comprising NaCl, sodium phosphate, pH 7.05, and maintained at stock concentrations of 14-50 mg/ml and stored at −70° C.

Aralast was maintained at stock concentrations of 20 mg/ml and stored at 4° C. A commercially available AAT (Prolastin™, Bayer) was used as a control. Recombinant human IL-18 was obtained from MBL International Corporation (Woburn Mass.). IL-6 and tumor necrosis factor (TNF) were obtained from R & D Systems, Minneapolis, Minn., endotoxin-free NaCl, and endotoxin (lipopolysaccharide, LPS) was obtained from Sigma (St. Louis, Mo.).

Medium for monocytic U1 cell and MAGI-CCR5 cell cultures consists of RPMI 1640 medium purchased from Mediatech (Herndon, Va.) containing 2.5 mM L-glutamine, 25 mM Hepes, 100 units/ml penicillin and 100 μg/ml streptomycin (GIBCO/BRL, Rockville, Md.) with 10% or 7.5% (v/v) heat-inactivated fetal bovine serum (FBS, GIBCO) for U1 cell and MAGI-CCR5 cell cultures, respectively. PBMC were cultured in R3 medium consisting of RPMI 1640 medium (Mediatech), 20% FBS (GIBCO), 100 units/ml penicillin and 100 μg/ml streptomycin (GIBCO) and 5% (vol/vol) IL-2 (Hemagen, Waltham, Mass.).

### U1 Monocytic Cell Assay

The U1 cell line was derived from human monocytic U937 cells into which 2 copies of HIV-1 provirus were incorporated into host genome. U1 cells were obtained from the AIDS Research and Reference Reagent Program, National Institute of Allergy and Infectious Diseases, NIH. U1 cells were maintained in T-175 polystyrene flasks (Falcon, Becton Dickinson, Franklin Lakes, N.J.) in medium and used when in log phase growth. Cells were counted in a hemacytometer, examined for viability by Trypan blue exclusion (>95% for all experiments) and resuspended in fresh medium at 2×10⁶ per ml. Two-hundred fifty μl of cell suspension were added to wells of 24-well polystyrene tissue culture plates (Falcon), followed by the addition of medium or AAT to produce the final concentration to be tested in a volume of 450 μl. After 1.0 hr of incubation (37° C., 5% CO₂), 50 μl of medium (control) or stimulus diluted in medium were added to wells to produce the final concentration of stimulus to be tested. The final culture volumes were 500 μl and contained 1×10⁶ cells per ml. After 48 hr of incubation (37° C. and 5% CO₂), 50 μl of 10% (v/v) Triton-X-100 (Fisher Scientific, Fair Lawn, N.J.) was added to each culture (final concentration of 1% v/v), and cultures frozen and thawed once. This was followed by assay for HIV-1 p24 antigen by ELISA with a lower limit of detection of 31 pg/ml (NCI-Frederick Cancer Research and Development Center, Frederick, Md.). The disruption of cells due to the addition of Triton-X-100 and the freeze-thaw cycle produced cell lysates and enabled assessment of total (secreted and cell-associated) production of p24 antigen.

### Peripheral Blood Mononuclear Cells (PBMC) Based HIV-1 Assay

PBMC from HIV-1 negative healthy subjects were isolated from heparinized blood by Ficoll-Hypaque gradient centrifugation. The concentration of PBMC in aliquots were counted using a hemacytometer (viability >95% by trypan blue exclusion for each experiment) and PBMC were diluted at 1×10⁶ per ml in R3 medium supplemented with additional 5% (v/v) IL-2 and 3.3 μg/ml phytohemagglutinin (PHA, Sigma). Cell suspensions were then incubated for 2 days (37° C., 5% CO₂) in T-175 polystyrene tissue culture flasks (Falcon) prior to infection with virus.

The stocks of lymphocyte-tropic HIV-1 strain A018A were titered by standard protocol and used to infect PBMC. Following the 2 days of incubation, PBMC from each donor were removed from tissue culture flasks, divided into 2 equal aliquots placed into 50 ml polypropylene tubes, concentrated by centrifugation and the medium decanted. Each parallel aliquot was infected by incubation with 300 tissue culture infective doses (TCID)₅₀ HIV-1 per 1×10⁶ cells for 3 hr in 500 μl medium. The two parallel PBMC infections from each donor were conducted in the absence or presence of 3 mg/ml AAT. The infected PBMC (without or with 3.0 mg/ml AAT) are then resuspended and washed in 15 ml R3 medium, pelleted, and resuspended at 2×10⁶ per ml in fresh R3 medium. Two hundred fifty μl of a suspension of HIV-1-infected PBMC were aliquoted into 24-well polystyrene tissue culture plates. An additional 250 μl R3 medium (control) or AAT was added to appropriate wells to produce a final culture volume of 500 μl containing 1×10⁶ cells per ml. For each donor, a separate 250 μl aliquot of PBMC suspension was added to a 1.5 ml polypropylene microfuge tube (Fisher) along with 200 μl R3 medium and 50 μl of 10% (vol/vol) Triton-X-100 (Fisher). This sample was frozen and designated time 0. Cultures in 24-well plates were incubated for 4 days, after which Triton-X-100 (Fisher) was added (final concentration of 1% v/v as described above for U1 cell cultures) and plates frozen and thawed once. Corresponding time 0 samples were thawed with each plate and cell lysates assayed for p24 antigen by ELISA.

### MAGI-CCR5 Cell Assay

The MAGI (Multinuclear Activation of a Galactosidase Indicator)-CCR-5 cell line is a clone derived from the HeLa cell line that expresses high levels of CD4. It has been transfected with a single integrated copy of a galactosidase gene under control of the HIV-1 long terminal repeat. β-Galactosidase is expressed upon production of HIV-1 Tat protein following one round of HIV-1 replication within the cell. The MAGI-CCR-5 cell line is derived from MAGI cells into which the CCR-5 HIV-1 co-receptor gene has been incorporated. These cells constitute an assay for early infection events and can be infected with either lymphocyte-tropic or macrophage-tropic HIV-1 strains.

MAGI-CCR-5 cells were obtained from the AIDS Research and Reference Reagent Program, National Institute of Allergy and Infectious Diseases, NIH. Cells were cultured in polystyrene T-175 flasks in medium until cells were noted to be in log growth phase. Cells were then resuspended in fresh medium and aliquoted into 24-well polystyrene plates at 4×10⁴ cells per well (1.0 ml total volume). After 24 hr incubation adherent cells were 30-40% confluent and medium was removed. Two hundred μl of fresh medium was then added to each well without (negative control) or with AAT and incubated for 1 hour. AAT diluent was added to a separate well at a volume equivalent to that of the highest concentration of AAT tested (control).

One hundred thirty TCID₅₀ of HIV-1 and DEAE dextran in medium were added to each well. T-cell tropic HIV-1 strain A018A was used. After 2 hr incubation, medium was added to each well to adjust the final volume of each well to 500 μl. Cultures were incubated for 48 hr, which allowed infection of the MAGI-CCR-5 cells. Medium was aspirated and the cells fixed for 5.0 min at room temperature by adding 1.0 ml of a 1% formaldehyde/0.2% glutaraldehyde solution in phosphate buffered saline (PBS). Fixing solution was then aspirated and cells washed with PBS. This was followed by addition of galactosidase staining solution. Fifty minutes of incubation was followed by a blinded optical count of pigmented cells under a microscope.

### Mice

C57BL/6 and DBA/2 female mice were purchased from Jackson Laboratories and housed under standard conditions.

### Induction of Hyperglycemia by Streptozotocin, Islet Isolation and Islet Transplantation.

5-6 weeks old C57BL/6 mice were treated intraperitoneally (i.p.) with 225 mg/kg Streptozotocin (STZ) (Sigma). Mice with established hyperglycemia were used at least 5 days after STZ administration. Islets were isolated from DBA/2 mice on day of transplantation, or 24 hours before in vitro assays, by enzymatic digestion of pancreatic tissue. Briefly, mice were anesthetized with i.p. ketamine (50 mg/kg, Vedco Inc.) and xylazine (10 mg/kg, Vedco Inc.) Each pancreas was inflated with 3.5 ml cold collagenase (1 mg/ml, type XI, Sigma), excised and immersed for 40 minutes at 37° C. in water bath. Pancreata were gently vortexed and filtered through 500-micron metal sieve. The pellet was washed twice in cold HBSS containing 0.5% BSA (Sigma) and reconstituted in RPMI-1640 (Cellgro, Mediatech) supplemented with 10% FCS (Cellgro), 50 IU/ml Penicillin (Cellgro) and 50 μg/ml streptomycin (Cellgro). Islets were collected on a 100-micron nylon cell strainer (BD Falcon), released into a petri dish by rinsing with HBSS (Cellgro, Mediatech) and 0.5% BSA (Sigma) and hand picked under a stereomicroscope. For transplantation, 450 islets were thoroughly washed from residual FCS in HBSS and 0.5% BSA and mounted on 0.2 ml tip for immediate transplantation. For in vitro assays islets were left to incubate for 24 hours at 37° C. Islet transplantation was performed into the left renal subcapsular space. Recipient mice were anesthetized, as described above. An abdominal wall incision was made over the left kidney. Islets were released into the subcapsular space through a puncture made in the capsule and the opening was sealed with a 1 mm³ block of sterile absorbable gelatin sponge (Surgifoam, Ethicon Inc.). Skin incision was closed with a 4-0 suture (USS/DG Sutures). Blood glucose follow-up was performed 3 times a week from end-tail blood drop using glucosticks (Roche).

### Development of Anti-Human-AAT Antibodies in Mice.

In order to evoke specific antibody production against human AAT, mice were injected i.p. with 10 mg human AAT per 20-gram mouse for four times in intervals of 1 week. Mice were used in experiments 2 months after last administration. Antibody production was evaluated before transplantation experiments were carried out.

### Assaying for Anti-Human-AAT Antibody Levels.

Briefly, mouse sera were kept at −70° C. until assayed for anti-human-AAT levels. Microtiter plates were coated with human AAT or albumin (2 μg/ml) in PBS at 4° C. overnight, then washed and blocked with blocking solution containing 10% BSA for 1 hour at 25° C. Wells were then washed and incubated with dilutions of test serum or negative control serum at 25° C. for 2 hours. After washing, goat-anti-mouse IgG-peroxide conjugate was used as secondary antibody (R&D, 1:1,000 dilution) to detect bound anti-AAT antibody using standard TMB substrate solution (Sigma).

### Cells.

NIH-3T3 cell line was purchased from ATCC and cultured in DMEM supplemented with 10% FCS, 50 IU/ml Penicillin and 50 μg/ml streptomycin. On day of peritoneal inoculation, 1×10⁷ cells were freshly collected by trypsinization and washed with cold PBS. Pellet was resuspended in 1 ml cold PBS for immediate injection.

### Infiltration Experiments.

Peritoneal infiltration was elicited by i.p. injection of 1 ml autoclaved thioglycolate (3% w/v, Sigma) or allogeneic cells (NIH-3T3), together with 0.1 ml saline, human albumin, human AAT or oxidized AAT. Peritoneal lavage was performed at 24 and 48 hours (thioglycolate) or on days 1-5 (allogeneic cells). For lavage, mice were anesthetized by isoflurane inhalation and injected immediately with 5.5 ml cold PBS containing 5% FCS and 5 U/ml heparin into the peritoneal cavity. After massaging the abdomen, peritoneal fluid was recovered with an 18.5 G needle and syringe. Recovered volumes ranged from 4.5 to 5.5 ml. Red blood cells were lysed (RBC lysing buffer, BD PharMingen) and cell counts were performed with a hemocytometer. Cells were then centrifuged and the pellet was resuspended in wash buffer containing 2% BSA, 0.1% sodium azide and 0.1% EDTA. Cells were kept at 4° C. throughout the staining procedure. Cells (at least 1×10⁶/polypropylene vial) were incubated with FcγRIII/II receptor blocking antibodies (Table 1) for 10 min. Cells were then divided into two groups and incubated with mAbs for leukocytes and either CD3/NK cells or neutrophil/monocytes/macrophages (Table I) for 30 min. Cells were washed twice with FACS wash buffer and fixed in 2% EM-grade formaldehyde. The number of cells expressing a particular marker was calculated by multiplying percentages obtained from flow-cytometry by the concentration of cells in lavage fluid.

### Insulin Immunohistochemistry.

AAT oxidation by myeloperoxidase (MPO) system. AAT (4 mg/ml) was incubated at 37° C. for 45 minutes with MPO (1 U/ml, Sigma), H₂O₂ (80 μM, Sigma) and NaCl (2.5 mM) in PBS, pH 7.4. Reaction was terminated by boiling for 1 hour followed by filter-centrifugation of MPO—H₂O₂ system products (Mw 30,000 cutoff, Centricon YM-30, Micron Bioseparations). Boiling was essential for the inactivation of MPO and did not inactivate AAT. Loss of activity of oxidized AAT was confirmed by elastase activity assay.

### Cytokine Assays.

Electrochemiluminescence (ECL) assays were used for the measurement of mouse TNFα and MIP-1α. Briefly, cytokine-specific goat anti-mouse affinity purified antibodies (R&D Systems, Minneapolis, Minn.) were labeled with ruthenium (BioVeris) according to manufacturer's instructions. Biotinylated polyclonal anti-mouse antibodies (R&D Systems) were diluted to a final concentration 1 μg/ml in PBS, pH 7.4, containing 0.25% BSA, 0.5% Tween-20, and 0.01% sodium azide (ECL buffer). Per assay tube, 25 μl of the biotinylated antibodies were incubated at room temperature with 25 μl of a 1 mg/ml solution of streptavidin-coated paramagnetic beads (Dynal Corp., Lake Success, N.Y.) for 30 min by vigorous shaking. Samples (25 μl) or standards that had been diluted in RPMI 1640 containing 5% FCS were added to tubes followed by 25 μl of ruthenilated antibody (final concentration 1 μg/ml, diluted in ECL buffer). The tubes were then shaken overnight. 200 μl of PBS was added per tube and the amount of chemiluminescence was determined using an Origen Analyzer (BioVeris).

### Membrane TNFα

Membrane TNFα on islet cells was detected by modification of a method for the evaluation of membrane TNFα on human PBMC. Briefly, single-cell suspension of islets was incubated with anti-mTNFα-PE mAb (Table 1). Cells were washed with FACS buffer and resuspended in 0.5 ml 2% EM-grade formaldehyde.

### Nitric Oxide Assay.

Nitrite levels in supernatants were determined using Griess reagent (Promega).

### Apoptosis Assay.

Freshly isolated human islets activate stress signaling pathways and exhibit high rates of apoptosis due to the process of isolation, necessitating the use of more than one islet donor per diabetic patient. It was observed that apoptosis that follows islet isolation was diminished when islets are cultured with AAT. In addition, islets that were cultured with AAT for 24 hours prior to transplantation were able to normalize serum glucose levels of diabetic mice when transplanted autologously at an otherwise sub-functional mass.

### AAT Dosage.

Normal human plasma typically contains 0.8-2.4 mg/ml of AAT, with a half life of 5-6 days. In gene transfer studies in C57BL/6 mice, plasma levels of 0.8-1.0 mg/ml were achieved and provided protection from type I diabetes in NOD mice. AAT administered intraperitoneally at 0.3-1.0 mg per mouse protected from TNFα-induced lethal response, and 0.8 mg AAT protected from D-galactosamine/LPS induced hepatic injury. Since AAT levels rise 3- to 4-fold during the acute phase response, 2 mg per mouse resulted in plasma levels that do not exceed physiological levels.

### Statistical Analysis

Data are presented as means±SEM. Group means were compared by two-sided t-test or ANOVA using Fisher's least significant difference for experiments with two or more subgroups. For data expressed as percent change, the values for p24 in control cultures (medium alone) were subtracted from those for each culture-containing stimulus. The p24 concentrations in cultures conducted in the presence of stimulus alone were set at 100%. Percent p24 in cultures containing stimulus and AAT were calculated by dividing the measured p24 by that present in cultures containing stimulus alone. The resultant fraction was expressed as a percent.

### Example 1

This example illustrates an, intracellular actin binding affect on HIV.

IL-6 was selected as a model pro-inflammatory and HIV-1-inducing cytokine. Whole blood was obtained from 3 healthy persons and cultured the blood for 24 hrs alone (undiluted) or diluted the blood with RPMI tissue culture medium to final blood concentrations of 1:4, 1:16, or 1:32. After 24 hrs of incubation (37° C., 5% CO₂), spontaneous IL-6 production was measured in the culture supernatants, and IL-6 concentrations were calculated per 1×10⁶ white blood cells (WBC). As shown in FIG. 1, whole blood dilution substantially increased IL-6 synthesis. These results suggest the presence of a natural suppressor in whole blood that blocks production of the HIV-1-inducing cytokine IL-6.

### Example 2

In this example, AAT was analyzed for inhibition of IL-6 in the blood.

AAT is a natural HIV-1 antagonist in whole blood. Whole blood was obtained and diluted the blood 1:16 with RPMI tissue culture medium alone or in the presence of 0.5 mg/mL AAT. After incubating for 24 hrs (37° C., 5% CO₂), culture supernatants were collected and IL-6 quantified as concentration per 1×10⁶ WBC. FIG. 2 shows the results of these experiments using blood from 3 healthy donors. As shown, AAT inhibited dilution-induced IL-6 production by approximately 50% (p<0.05). Therefore, it is believed that AAT blocks production of HIV-1-inducing IL-6 in blood.

Blood contains potent natural inhibitors of pro-inflammatory cytokines, and AAT is believed to be one of such inhibitors. IL-6 is a known inducer of HIV-1 production in vitro. It appears inhibition of IL-6 production by AAT is a mechanism by which AAT inhibits HIV-1 production in infected persons. Furthermore, AAT also appears to inhibit the pro inflammatory and HIV-1 inducing cytokines IL (interleukin)-1 and TNFα₂.

### Example 3

This example illustrates a method for biotinylating AAT.

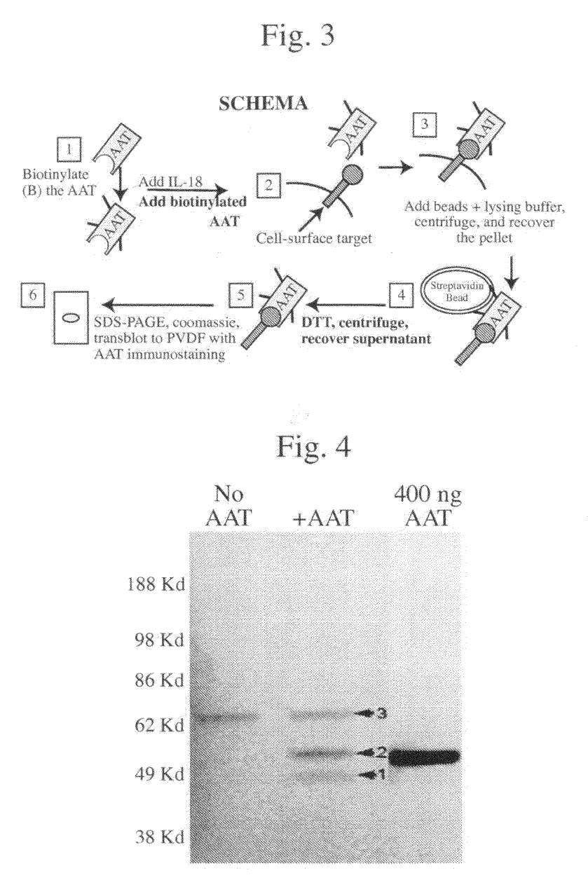

Based on mass spectroscopy isolation, it has been shown that AAT binds to cell surfaces. In the isolation strategy (FIG. 3), clinical grade AAT purified from the pooled plasma of healthy donors was used. This AAT preparation was developed for intravenous injection into persons with AAT deficiency (Aralast®, Baxter). For these experiments, AAT was biotinylated using the EZ Link Sulfo NHS SS Biotin kit (Pierce), as shown in the FIG. 3. This reagent biotinylates terminal amines and lysine residues and contains a short reducible linker that allows separation of the biotinylated complexes from a solid phase. The biotinylated AAT product was separated from the unbound biotinylating reagent using a desalting column (Pierce) equilibrated with PBS buffer. The purified biotinylated AAT was quantified using a bicinchinonic acid assay (Pierce).

### Example 4

This example illustrates a method for identifying a protein that AAT binds to.

To identify the AAT-binding protein present on host cell surfaces, fresh PBMC were stimulated for 2 days with IL-2 and phytohemaglutinin (PHA) to replicate conditions used for HIV-1 infection of PBMC. The cells were centrifuged, washed with PBS, and resuspended in serum-free RPMI medium. Biotinylated AAT (0.2 mg/mL) was added to the culture and the cells incubated at 37° C. for 6 hr to allow binding of the AAT to the cell surface target (label 2 in FIG. 3). Streptavidin beads were added to the cell suspension (ImmunoPure Immobilized Streptavidin Gel, Pierce) to bind the cell surface biotinylated AAT protease complexes (label 3 in FIG. 3).

The cell suspensions were then subjected to lysis to separate the bead biotinylated AAT protease complexes from other cell constituents. To accomplish this lysing buffer was used consisting of 0.15 M NaCl, 10 mM Hepes, 4 mM EDTA, 2.5% (w/v) Nonidet NP40 (NP40: Calbiochem), 2.5% (w/v) Zwittergent 3 14 (SB14, Calbiochem), 2.0 mg/ml BSA (bovine serum albumin crystallized, fatty acid free, Sigma), 0.05% NaN₃, and 200 μM phenylmethylsulfonylfluoride, pH 7.5. The PBMC were suspended in 750 μl of the lysing buffer at 4° C. for 30 minutes. Centrifugation and removal of the supernatant yielded a pellet containing streptavidin beads bound to complexes consisting of biotinylated AAT and attached target (label 4 in FIG. 3). The pellets were washed twice in PBS, and then 50 mM dithiothreitol (DTT, Pierce) was added to sever the reducible linker present in the biotinylation reagent (label 5 in FIG. 3). This process separated the protein complexes from the beads. Centrifugation and recovery of the supernatant produced the AAT protease complexes. The released protein complexes were electrophoresed into a denaturing SDS-PAGE gel and stained for the presence of complexes using colloidal coomassie (label 6 in FIG. 3).

In a parallel control experiment, the above protocol was followed except PBMC were cultured in the absence of AAT. This allowed assessment of non AAT protein adherence to the streptavidin beads. Results from the target isolation experiments are represented in FIG. 4. The far left lane (No AAT) shows results for PBMC cultured without added AAT, the center lane (+AAT) shows results for PBMC cultured in the presence of AAT, and the far right lane (400 ng AAT) shows 400 ng AAT alone run into the gel as a separate control. In the experimental center lane (+AAT), 3 distinct bands were visualized in the gel that represented separate proteins comprising the AAT-containing complexes. The complexed proteins separated when the complexes were subjected to the denaturing conditions of SDS-PAGE. The components of the complexes are represented by these 3 bands. Band 3 appeared in both the control experiment (No AAT) and in the experiment where AAT was added to the cultures (+AAT). It is believed that this band represents a non-specific protein purified from cells using the streptavidin beads. Band 2 is believed to be AAT because had identical migration as the AAT control (400 ng AAT). Band 1 migrated uniquely in the gel and was purified for further analysis. After excising band 1 from the gel, matrix-assisted laser desorption ionization time-of-flight (MALDI-TOF) mass spectroscopy was used to identify this protein. MALDI-TOF analysis identified band 1 as actin. Monomeric actin has a mass of 42 Kd, similar to the mass of band 1 (molecular mass markers are indicated to the left of the gel). This experiment demonstrated a physical association between AAT and monomeric actin on PBMC cell surfaces.

### Example 5

This example illustrates that AAT induces actin depolymerization in human cells cultured in vitro.

The effect of AAT on actin cytoskeletal structure was assessed using a direct imaging approach. HeLa cells were incubated with 0, 4 or 6 mg/mL of AAT for 24 hr and the intracellular actin cytoskeleton was visualized using phalloidin, a fluorescent chemical which binds and labels actin. Control cells (No AAT) incubated in medium alone had a clearly defined and well-ordered actin cytoskeleton, where actin was stained red and nuclei blue (data not shown). When these cells were incubated with increasing concentrations of AAT, marked disruption of intracellular cytoskeletal architecture was observed. At 4 mg/mL of AAT, fragmentation and disorganization of actin filaments was observed, for example, the presence of AAT resulted in a partially collapsed cytoskeleton (data not shown). At 6 mg/mL of AAT, many of the cells were unable to stick to the culture slide and most actin filaments were ablated. No evidence of AAT toxicity was detected at each of these physiologically-relevant concentrations. These results indicate that AAT disrupts the intracellular actin cytoskeleton without causing cytotoxicity. It is possible that a signal is generated inside the cell after AAT docks with cell surface actin, similar to intracellular signaling that ensues after a cytokine binds to its extracellular receptor. Regardless of its mechanism, it is believed that AAT disrupted the intracellular actin.

Cytochalasin D (cyto D) disrupts intracellular actin polymerization to inhibit HIV-1 infection of cells. It is believed that cyto D blocks the movement of viral co-receptors on the cell surface. This cyto D effect immobilizes HIV-1 co-receptors and blocks co-binding of HIV-1 to CD4 and a chemokine co-receptor (CCR5 and/or CXCR4) that enables HIV-1 entry into a cell. Actin disruption inhibits intracellular transport of maturing viral particles, because physical association with actin filaments is believed to be important to move these large molecular species within the cell cytoplasm.

### Example 6

This example shows that AAT inhibits actin polymerization in a cell free system.

The actin disrupting effect of AAT was demonstrated using an in vitro assay that quantifies actin polymerization in real time. Actin monomers were labeled with pyrene, which fluoresce when exposed to UV light. When actin monomers polymerize, the magnitude of the fluorescence signal increases and corresponds to the degree of actin polymerization.

Polymerization of actin was catalyzed by the addition of vesicles isolated from the yeast Saccharomyces cerivisiae. As shown in FIG. 5 (representative of 4 experiments), actin polymerization increased over time in the Control experiment (no AAT added). However, the addition of 1.3 mg/mL of AAT substantially inhibited actin polymerization. AAT reduced actin polymerization in both cultured cells and in a cell free system.

### Example 7

This example shows that AAT inhibits production of HIV-1 in U1 cell cultures.

Exposing U1 cells to pro-inflammatory cytokines such as IL-18, IL-1, IL-6 and TNF, phorbol esters or hyperosmolarity results in the induction of HIV-1 as assessed by p24 antigen. Stimulation of U1 cells with 0.5 nM of IL-18 induced large amounts of p24 antigen after 48 hr of incubation in 3 separate experiments. U1 cells cultured in medium alone (control) contained a mean of 41.3±11.5 pg/ml of p24 antigen, which increased 150-fold to 6,235±1,775 pg/ml of p24 following stimulation with IL-18. Cultures conducted in the presence of AAT added 1 hour prior to the addition of IL-18 demonstrated a dose-dependent reduction in p24 after 48 hours of culture, with near ablation of IL-18-induced p24 observed at 3 mg/ml of AAT. AAT added at 0.1, 0.5, 1, 2 and 3 mg/ml resulted in 6,879±207, 3,687±968, 2,029±625, 452±209 and 179±79 pg/ml of p24 production, respectively. At 1, 2 and 3 mg/ml of AAT, the percent reductions observed compared to stimulation with IL-18 alone were 65±1.8, 93±3.0 and 98±1%, respectively.

To evaluate the effect of AAT on U1 cell proliferation and viability, three experiments were performed in the presence or absence of 5 mg/ml of AAT. U1 cells were added at 1×10⁶ cells per ml and cultured for 48 hrs. Following incubation, cells were quantified using a hemacytometer. The mean±SEM cell concentrations in control and AAT-containing cultures were 2.5×10⁶±0.5×10⁶ and 2.4×10⁶±0.3×10⁶, respectively. These values were significantly higher than the 1×10⁶ cells per ml added initially (P<0.05), but they were not significantly different from one another. For all cultures, cell viability by trypan blue exclusion was >95%.

In 4 separate experiments, using 100 ng/ml of IL-6 as a stimulus, the mean p24 antigen measured in U1 cells cultured in medium alone (control) was 1,207±361 pg/ml. Stimulation with 100 ng/ml of IL-6 resulted in a 3.6-fold increase in p24 antigen production, to 4,337±2,006 pg/ml. Stimulation with IL-6 in the presence of AAT resulted in dose-dependent inhibition of p24 production compared to that measured in the absence of AAT. With the addition of AAT at 0.1, 0.5, 1, 2, 3, 4, and 5 mg/ml, the measured P24 antigen values were 6,228±2,129, 3,992±1,987, 3,850±1,943, 2,597±1,253, 2,155±1,085, 1,838±881 and 1,213±658 pg/ml, respectively. The corresponding mean percent reductions for AAT additions of 3, 4 and 5 mg/ml were 80, 88 and 100%, respectively.

In several (e.g. 4) separate experiments where U1 cells were exposed to TNF as stimulus, the mean p24 antigen measured in control and TNF-stimulated (3.0 ng/ml) cultures were 2,328±1,680 and 18,635±5,243 pg/ml, respectively. This 8-fold increase in p24 production was significantly and dose-dependently reduced in the presence of AAT. Inclusion of AAT at the concentrations 0.1, 0.5, 1, 2, 3, 4, and 5 mg/ml reduced TNF-induced p24 antigen to 16,405±8,449, 16,863±7,718, 15,328±7,129, 12,566±4,981, 9,341±2,730, 9,091±3,436 and 6,868±2,737, respectively. The mean percent reductions in TNF-induced p24 antigen observed in the presence of 3, 4, and 5 mg/ml of AAT were 56, 60, and 73%, respectively.

LPS is a cell wall component of gram-negative bacteria with several pro-inflammatory activities. In 3 experiments, U1 cells cultured in the presence of 500 ng/ml of LPS for 48 hrs contained 1,427±39 pg/ml p24 antigen. This represents a mean 3-fold increase compared to p24 produced in control (medium alone) cultures, where 476±76 pg/ml p24 antigen was measured. U1 cells stimulated with LPS in the presence of 0.1, 0.5, 1, 2, 3, 4, and 5 mg/ml of AAT contained 1,531,±436, 1,543,±427, 1,108±241, 913±287, 782,±187, 578,±155, and 626±257 pg/ml of p24 antigen, respectively. Addition of AAT at 3, 4, and 5 mg/ml inhibited p24 production by 71, 90 and 86%, respectively.

### Example 8

This example shows that AAT inhibits NaCl-induced HIV-1 in U1 cell cultures.

To exclude the possibility that AAT-induced inhibition of cytokine-stimulated p24 was due to extracellular (supernatant) protein-protein interactions, hyperosmolarity was used as the p24-inducing stimulus. A 60 mM solution of NaCl is a potent inducer of p24 antigen in U1 cell cultures. Three experiments were conducted to test the effect of AAT on NaCl-induced p24. A large (26-fold) increase in mean p24 antigen production in cultures was observed in the presence of NaCl alone as compared to control (medium alone) cultures. The mean p24 antigen measured in NaCl-stimulated and control cultures were 7,511±707 and 295±29 pg/ml, respectively. Stimulation with 60 mM of NaCl solution in the presence of 0.1, 0.5, 1.0, 2.0, 3.0, 4.0 and 5.0 mg/ml of AAT resulted in mean p24 levels of 11,054±3,231, 7,363±485, 5,657±48, 2,83 8±466, 1,919±594, 425±32 and 266±26 pg/ml, respectively. For AAT added at 3.0, 4.0 and 5.0 mg/ml, the corresponding percent inhibitions were 76, 98.3 and 100%.

### Example 9

This example shows that AAT inhibits p24 antigen production in HIV-1-infected PBMC.

The effect of AAT on freshly-infected PBMC was tested to assess activity in a primary cell model of HIV-1 infection. PBMC isolated from 3 healthy volunteers were infected with lymphocyte-tropic HIV-1 as described above. Results obtained for PBMC infected with HIV-1 in the presence of 3 mg/ml of AAT at the time of infection showed that a large increase in p24 antigen occurred over the 4 days of culture.

Compared to time 0, a significant increase in p24 production was observed in control cultures after 4 days of culture, with values of 107±52 and 8,478±629 pg/ml, respectively (mean 79-fold increase, P<0.001). PBMC cultured in the presence of AAT added at 0.1, 0.5, 1.0, 2.0, 3.0, 4.0 and 5.0 mg/ml produced 6,620±2,026, 6,047±1,322, 6,014±2,055, 2,516±345, 3,360±371, 2,743±316 and 2,713±645 pg/ml, respectively. Significant reductions in p24 antigen in cultures exposed to AAT compared to control cultures were observed for AAT concentrations of 2.0, 3.0, 4.0 and 5.0 mg/ml. Compared to control cultures, these AAT concentrations resulted in reductions in p24 production of 71, 61, 65 and 67%, respectively.

### Example 10

This example shows that AAT inhibits early infection-associated events in MAGI-CCR5 cells exposed to HIV-1.

The MAGI-CCR-5 cell assay evaluates early events in the HIV-1 infection process. These events include cell-surface binding and internalization, uncoating, reverse transcription and translation, protein processing and Tat activity. Binding of the tat protein to a reporter construct within the MAGI-CCR-5 cells enables quantification of these early HIV-1 events.

In three separate experiments, MAGI-CCR-5 cells were infected with A018A strain of HIV-1. In cultures conducted in the absence of virus (no HIV-1), a mean positive cell count of 2.3 was obtained. In the presence of HIV-1 (+HIV-1), an increase in mean positive cell count was observed (72±13, 31-fold increase, P<0.001). MAGI-CCR-5 cells exposed to HIV-1 and cultured with added AAT demonstrated significant and dose-dependent inhibition of positive cell counts. Addition of 0.1, 1.0, 2.0, 3.0, 4.0 and 5.0 mg/ml of AAT resulted in mean positive cell counts of 74±13, 75±17, 56±11, 45±12, 28±9, and 21±12, respectively.

Compared to cultures containing HIV-1 alone, inhibition of MAGI-CCR-5 cell early infection events was significant for AAT concentrations of 2.0, 3.0, 4.0 and 5.0 mg/ml. These values correspond to 23, 41, 66 and 76% inhibition. As a vehicle control, MAGI-CCR-5 cells were exposed to virus and a diluent volume equivalent to that of AAT solution added to 5.0 mg/ml cultures. Cultures containing diluent produced a positive cell count of 72±16, which was not significantly different from cultures containing HIV-1 alone (+HIV).

### Example 11

This example shows the effect of potential intracellular actin binding peptides on HIV-1.

A carboxyterminal peptide FVYLI (SEQ ID NO: 16) was analyzed for its effect on HIV-1. Other short peptides such as FVFLM (SEQ ID NO: 1), FVFAM (SEQ ID NO:2), FVALM (SEQ ID NO:3), FVFLA (SEQ ID NO:4), FLVFI (SEQ ID NO:5), FLMII (SEQ ID NO:6), FLFVL (SEQ ID NO:7), FLFVV (SEQ ID NO:8), FLFLI (SEQ ID NO:9), FLFFI (SEQ ID NO:10), FLMFI (SEQ ID NO:11), FMLLI (SEQ ID NO:12), FIIMI (SEQ ID NO: 13), FLFCI (SEQ ID NO: 14), FLFAV (SEQ ID NO: 15), FVYLI (SEQ ID NO: 16), FAFLM (SEQ ID NO: 17), AVFLM (SEQ ID NO: 18) demonstrated a similar effect. They were active at approximately similar molar range in the MAGI cultures when used alone or in combination. This shows that peptides derived from or homologous and/or analogous to this C-terminal region of AAT are antivirally active.

### Example 12

This example shows anti-HIV-1 effect of drugs having extracellular actin binding activity.

A series of drugs that have intracellular actin binding activity were tested for anti-HIV-1 activity. These synthetic drugs were made according to methods described in WO 98/24806, which is incorporated herein by reference in its entirety. Briefly, WO 98/24806 discloses substituted oxadiazole, thiadiazole and triazole compounds. In addition, U.S. Pat. No. 5,874,585 discloses substituted heterocyclic compounds; U.S. Pat. No. 5,869,455 discloses N-substituted derivatives; U.S. Pat. No. 5,861,380 discloses keto and di-keto containing ring systems; U.S. Pat. No. 5,807,829 discloses tripeptoid analogues; U.S. Pat. No. 5,801,148 discloses proline analogues; U.S. Pat. No. 5,618,792 discloses substituted heterocyclic compounds.

Surprisingly, several of these drugs demonstrate anti-HIV-1 activity at micromolar ranges. A synthetic molecule (P3 inh) displayed significant anti-HIV-1 effect in the same experimental condition as in Example 7 and the experimental conditions disclosed in Shapiro, et al, FASEB J, 2001 January; 15 (1):115-122. P3 inh is also known as CE-2072 or (benzyloxycarbonyl)-L-valyl-N-[1-(2-(3-methylbenzyl)-1,3,4-oxadiazolyl]carbonyl)-2-(S)-methylpropyl]-L-prolinamide. Methods of preparing P3 inh and its derivatives are disclosed in U.S. Pat. No. 5,807,829, which is incorporated by reference herein. CE 2072 along with AAT were tested in an assay that demonstrates the effect of these substances on NF-κB expression, which is induced by IL-18. A band that corresponds to IL-18-induced NF-κB was much larger than NF-κB in controls not stimulated by IL-18. In the presence of either AAT or AAT-mimicking synthetic molecule, the NF-κB expression was reduced, indicating that these substances down-regulate NF-κB expression. This was an unexpected observation as these compounds are not known to interfere with NF-κB expression. In addition, other experiments have demonstrated that AAT inhibits NF-κB activation.

### Example 13

This example shows antiviral activity of synthetic actin modulators.

Compounds such as, but are not limited to, oxadiazole, thiadiazole and triazole peptoids can also be used in methods of the invention as these compounds show an antiviral activity. Anti-HIV-1 effective doses were in a range from about 1 μg/kg to approximately 100 mg/kg. Specific examples of such oxadiazole, thiadiazole and triazole peptoids are compounds such as Benzyloxycarbonyl-L-valyl-N-[1-(2-(3-methylbenzyl)-1,3,4-oxadiazolyl]carbonyl)-2-(S)-methylpropyl]-L-prolinamide; Benzyloxycarbonyl-L-valyl-N-[1-(2-(5-(methyl)-1,3,4-oxadiazoly]carbony)-2-(S)-methylpropyl]-L-prolinamide; Benzyloxycarbonyl)-L-valyl-N-[1-(2-(5-(3-trifluoromethylbenzyl)-1,3,4-oxadiazolyl]carbonyl)-2-(S)-methylpropyl]-L-prolinamide Benzyloxycarbonyl)-L-valyl-N-[1-(2-(5-(4-Dimethylaminobenzyl)-1,3,4-oxadiazolyl]carbonyl)-2-(S)-methylpropyl]-L-prolinamide; Benzyloxycarbonyl)-L-valyl-N-[1-(2-(5-(1-napthylenyl)-1,3,4-oxadiazolyl]carbonyl)-2-(S)-methylpropyl]-L-prolinamide; (Benzyloxycarbonyl)-L-valyl-[1-(3-(5-(3,4-methylenedioxybenzyl)-1,2,4-oxadiazolyl]carbonyl)-2-(S)-methylpropyl]-L-prolinamide; Benzyloxycarbonyl)-L-valyl-N-[1-(3-(5-(3,5-dimethylbenzyl)-1,2,4-oxadiazolyl]carbonyl)-2-(S)-methylpropyl]-L-prolinamide; (Benzyloxycarbonyl)-L-valyl-N-[1-(3-(5-(3,5-dimethoxybenzyl)-1,2,4-oxadiazolyl]carbonyl)-2-(S)-methylpropyl]-L-prolinamide; (Benzyloxycarbonyl)-L-valyl-N-[1-(3-(5-(3,5-ditrifluoromethylbenzyl)-1,2,4-oxadiazolyl]carbonyl)-2-(S)-methylpropyl]-L-prolinamide; (Benzyloxycarbonyl)-L-valyl-N-[1-(3-(5-(3-methylbenzyl)-1,2,4-oxadiazolyl]carbonyl)-2-(S)-methylpropyl]-L-prolinamide; (Benzyloxycarbonyl)-L-valyl-N-[1-(3-(5-(biphenylmethine)-1,2,4-oxadiazolyl]carbonyl)-2-(S)-methylpropyl]-L-prolinamide; (Benzyloxycarbonyl)-L-valyl-N-[1-(3-(5-(4-phenylbenzyl)-1,2,4-oxadiazolyl]carbonyl)-2-(S)-methylpropyl]-L-prolinamide; (Benzyloxycarbonyl)-L-valyl-N-[1-(3-(5-(3-phenylbenzyl)-1,2,4-oxadiazolyl]carbonyl)-2-(S)-methylpropyl]-L-prolinamide; (Benzyloxycarbonyl)-L-valyl-N-[1-(3-(5-(3-phenoxybenzyl)-1,2,4-oxadiazolyl]carbonyl)-2-(S)-methylpropyl]-L-prolinamide; (Benzyloxycarbonyl)-L-valyl-N-[1-(3-(5-(cyclohexylmethylene)-1,2,4-oxadiazolyl]carbonyl)-2-(S)-methylpropyl]-L-prolinamide; (Benzyloxycarbonyl)-L-valyl-N-[1-(3-(5-(3-trifluoromethyldimethylmethylene)-1,2,4-oxadiazolyl]carbonyl)-2-(S)—methylpropyl]-L-prolinamide; (Benzyloxycarbonyl)-L-valyl-N-[1-(3-(5-(1-napthylmethylene)-1,2,4-oxadiazolyl]carbonyl)-2-(S)-methylpropyl]-L-prolinamide; (Benzyloxycarbonyl)-L-valyl-N-[1-(3-(5-(3-pyridylmethyl)-1,2,4-oxadiazolyl]carbonyl)-2-(S)-methylpropyl]-L-prolinamide; (Benzyloxycarbonyl)-L-valyl-N-[1-(3-(5-(3,5-diphenylbenzyl)-1,2,4-oxadiazolyl]carbonyl)-2-(S)-methylpropyl]-L-prolinamide; (Benzyloxycarbonyl)-L-valyl-N-[1-(3-(5-(4-dimethylaminobenzyl)-1,2,4-oxadiazolyl]carbonyl)-2-(S)-methylpropyl]-L-prolinamide; 2-(5-[(Benzyloxycarbonyl)amino]-6-oxo-2-(4-fluorophenyl)-1,6-dihydro-1-pyrimidinyl]-N-[1-(3-(5-(3-trifluoromethylbenzyl)-1,2,4-oxadiazolyl]carbonyl)-(S)-2-methylpropyl]acetamide; 2-(5-Amino-6-oxo-2-(4-fluorophenyl)-1,6-dihydro 1-pyrimidinyl]-N-[1-(3-(5-(3-trifluoromethylbenzyl)-1,2,4-oxadiazolyl]carbonyl)-2-(S)-methylpropyl]acetamide; 2-(5-[(Benzyloxycarbonyl)amino]-6-oxo-2-(4-fluorophenyl)-1,6-dihydro-1-pyrimidinyl]-N-[1-(2-(5-(3-methylbenzyl)-1,3,4-oxadiazolyl]carbonyl)-(S)-2-methylpropyl]acetamide; 2-(5-Amino-6-oxo-2-(4-fluorophenyl)-1,6-dihydro-1-pyrimidinyl]-N-[1-(2-(5-(3-methylbenzyl)-1,3,4-oxadiazolyl]carbonyl)-2-methylpropyl]acetamide; (Pyrrole-2-carbonyl)-N-(benzyl)glycyl-N-[1-(2-(5-(3-methylbenzyl)-1,3,4-oxadiazolyl]carbonyl)-2-(S)-methylpropyl]amide; (Pyrrole-2-carbonyl)-N-(benzyl)glycyl-N-[1-(3-(5-(3-trifluoromethylbenzyl)]-1,2,4-oxadiazolyl)-(S)-methylpropyl]amide; (2S,5S)-5-Amino-1,2,4,5,6,7-hexahydroazepino-[3,2,1]-indole-4-one-carbonyl-N-[1-(2-(5-(3-methylbenzyl)-1,3,4-oxadiazolyl]carbonyl)-(R,S)-2-methylpropyl]amide; BTD-[1-(2-(5-(3-methylbenzyl)-1,3,4-oxadiazolyl]carbonyl)-2-(S)-methylpropyl]amide; (R,S)-3-Amino-2-oxo-5-phenyl-1,4,-benzodiazepine-N-[1-(2-(5-(3-methylbenzyl)-1,3,4-oxadiazolyl]carbonyl)-2-(S)-methylpropyl]acetamide; (Benzyloxycarbonyl)-L-valyl-2-L-(2,3-dihydro-1H-indole)-N-[1-(2-(5-(3-methylbenzyl)-1,3,4-oxadiazolyl]carbonyl)-2-(S)-methylpropyl]amide; (Benzyloxycarbonyl)-L-valyl-2-L-(2,3-dihydro-1H-indole)-N-[1-(3-(5-(3-trifluoromethylbenzyl)-1,2,4-oxadiazolyl]carbonyl)-2-(S)-methylpropyl]amide; Acetyl-2-L-(2,3-dihydro-1H-indole)-N-[1-(2-(5-(3-methylbenzyl)-1,3,4-oxadiazolyl]carbonyl)-2-(S)-methylpropyl]amide; 3-(S)-(Benzyloxycarbonyl)amino)-.epsilon.-lactam-N-[1-(2-(5-(3-methylbenzyl)-1,3,4-oxadiazolyl]carbonyl)-2-(S)-methylpropyl]acetamide; 3-(S)-(Amino)-.epsilon.-lactam-N-[1-(2-(5-(3-methylbenzyl)-1,3,4-oxadiazolyl]carbonyl)-2-(S)-methylpropyl]acetamide trifluoroacetic acid salt; 3-(S)-[(4-morpholinocarbonyl-butanoyl)amino]-.epsilon.-lactam-N-[1-(2-(5-(3-methylbenzyl)-1,3,4-oxadiazolyl]carbonyl)-2-(R,S)-methylpropyl]acetamide; 6-[4-Fluorophenyl]-.epsilon.-lactam-N-[1-(2-(5-(3-methylbenzyl)-1,3,4-oxadiazolyl]carbonyl)-2-(S)-methylpropyl]acetamide; 2-(2-(R,S)-Phenyl-4-oxothiazolidin-3-yl]-N-[1-(2-(5-(3-methylbenzyl)-1,3,4-oxadiazolyl]carbonyl)-2-(S)-methylpropyl]acetamide; 2-(2-(R,S)-phenyl-4-oxothiazolidin-3-yl]-N-[1-(2-(5-(3-methylbenzyl)-1,3,4-oxadiazolyl]hydroxymethyl)-2-(S)-methylpropyl]acetamide; 2-(2-(R,S)-Benzyl-4-oxothiazolidin-3-yl]-N-[1-(2-(5-(3-methylbenzyl)-1,3,4-oxadiazolyl]carbonyl)-2-(S)-methylpropyl]-acetamide; 2-(2-(R,S)-Benzyl-4-oxothiazolidin-3-yloxide]-N-[1-(3-(5-(3-trifluoromethylbenzyl)-1,2,4-oxadiazolyl]carbonyl)-2-(R,S,)-methylpropyl]acetamide; (1-Benzoyl-3,8-quinazolinedione)-N-[1-(2-(5-(3-methylbenzyl)-1,3,4-oxadiazolyl]carbonyl)-2-(S)-methylpropyl]acetamide; (1-Benzoyl-3,6-piperazinedione)-N-[1-(2-(5-(3-methylbenzyl)-1,3,4-oxadiazolyl]carbonyl)-2-(S)-methylpropyl]acetamide; (1-Phenyl-3,6-piperazinedione)-N-[1-(2-(5-(3-methylbenzyl)-1,3,4-oxadiazolyl]carbonyl)-2-(S)-methylpropyl]acetamide; [(1-Phenyl-3,6-piperazinedione)-N-[1-(3-(5-(3-trifluoromethylbenzyl)-1,2,4-oxadiazolyl]carbonyl)]-2-(S)-methylpropyl]acetamide; 3-[(Benzyloxycarbonyl)amino]-quinolin-2-one-N-[1-(2-(5-(3-methybenzyl)-1,3,4-oxadiazolyl]carbonyl)-2-(S)-methylpropyl]acetamide; 3-[(Benzyloxycarbonyl)amino]-7-piperidinyl-quinolin-2-one-N-[1-(2-(5-(3-methybenzyl)-1,3,4-oxadiazolyl]carbonyl)-2-(S)-methylpropyl]acetamide; 3-(Carbomethoxy-quinolin-2-one-N-[1-(2-(5-(3-methybenzyl)-1,3,4-oxadiazolyl]carbonyl)-2-(S)-methylpropyl]acetamide; 3-(Amino-quinolin-2-one)-N-[1-(2-(5-(3-methylbenzyl)-1,3,4-oxadiazolyl]carbonyl)-2-(S)-methylpropyl]acetamide; 3-[(4-Morpholino)aceto]amino-quinolin-2-one-N-[1-(2-(5-(3-methylbenzyl)-1,3,4-oxadiazolyl]carbonyl)-2-(S)-methylpropyl]acetamide; 3,4-Dihydro-quinolin-2-one-N-[1-(2-(5-(3-methylbenzyl)-1,3,4-oxadiazolyl]carbonyl)-2-(S)-methylpropyl]acetamide; 1-Acetyl-3-(4-fluorobenzylidene)piperazine-2,5-dione-N-[1-(2-(5-(3-methylbenzyl)-1,3,4-oxadiazolyl]carbonyl)-2-(S)-methylpropyl]acetamide; 1-Acetyl-3-(4-dimethylaminobenzylidene)piperazine-2,5-dione-N-[1-(2-(5-(3-methylbenzyl)-1,3,4-oxadiazolyl]carbonyl)-2-(S)-methylpropyl]acetamide; 1-Acetyl-3-(4-carbomethoxybenzylidene)piperazine-2,5-dione-N-[1-(2-(5-(3-methylbenzyl)-1,3,4-oxadiazolyl]carbonyl)-2-(S)-methylpropyl]acetamide; 1-Acetyl-3-[(4-pyridyl)methylene]piperazine-2,5-dione-N-[1-(2-(5-(3-methylbenzyl)-1,3,4-oxadiazolyl]carbonyl)-2-(S)-methylpropyl]acetamide; 4-[1-Benzyl-3-(R)-benzyl-piperazine-2,5,-dione]-N-[1-(2-[5-(3-methylbenzyl)-1,3,4-oxadiazolyl]carbonyl)-2-(S)-methylpropyl]acetamide; 4-[1-Benzyl-3-(S)-benzyl piperazine-2,5,-dione]-N-[1-(2-(5-(3-methylbenzyl)-1,3,4-oxadiazolyl]carbonyl)-2-(S)-methylpropyl]acetamide; 4-[1-Benzyl-3(R)-benzylpiperazine-2,5,-dione]-N-[1-(3-(5-(3-trifluoromethylbenzyl)-1,2,4-oxadiazolyl]carbonyl)-2-(S)-methylpropyl]acetamide; 4-[1-Benzyl-3-(S)-benzylpiperazine-2,5,-dione]-N-[1-(3-(5-(3-trifluoromethylbenzyl)-1,2,4-oxadiazolyl]carbonyl)-2-(S)-methylpropyl]acetamide; 4-[1-Benzyl-3-(S)-benzyl piperazine-2,5,-dione]-N-[1-(3-(5-(2-dimethylaminoethyl)-1,2,4-oxadiazolyl]carbonyl)-2-(S)-methylpropyl]acetamide; 4-[1-Methyl-3-R,S)-phenylpiperazine-2,5,-dione]-N-[1-(3-(5-(3-trifluoromethylbenzyl)-1,2,4-oxadiazolyl]carbonyl)-2-(S)-methylpropyl]acetamide; 4-[1-Methyl-3-(R,S)-phenylpiperazine-2,5,-dione]-N-[1-(2-(5-(3-methylbenzyl)-1,3,4-oxadiazolyl]carbonyl)-2-(S)-methylpropyl]acetamide; 4-[1-(4-Morpholinoethyl)3-(R)-benzylpiperazine-2,5,-dione]-N-[1-(2-(5-(3-methylbenzyl)-1,3,4-oxadiazolyl]carbonyl)-2-(S)-methylpropyl]acetamide; 5-(R,S)-Phenyl-2,4-imidazolidinedione-N-[1-(2-(5-(3-methylbenzyl)-1,3,4-oxadiazolyl]carbonyl)-2-(S)-methylpropyl]acetamide; 5-(R)-Benzyl-2,4-imidazolidinedione-N-[1-(2-(5-(3-methylbenzyl)-1,3,4-oxadiazolyl]carbonyl)-2-(S)-methylpropyl]acetamide; 5-(S)-Benzyl-2,4-imidazolidinedione-N-[1-(2-(5-(3-methylbenzyl)-1,3,4-oxadiazolyl]carbonyl)-2-(S)-methylpropyl]acetamide; 5-(S)-Benzyl-2,4-imidazolidinedione-N-[1-(3-(5-(3-trifluoromethylbenzyl)-1,2,4-oxadiazolyl]carbonyl)-2-(S)-methylpropyl]acetamide; 5-(R)-Benzyl-2,4-imidazolidinedione-N-[1-(3-(5-(3-trifluoromethylbenzyl)-1,2,4-oxadiazolyl]carbonyl)-2-(S)-methylpropyl]acetamide; 1-Benzyl-4-(R)-benzyl-2,5-imidazolidinedione-N-[1-(2-(5-(3-methylbenzyl)-1,3,4-oxadiazolyl]carbonyl)-2-(S)-methylpropyl]acetamide; and 1-Benzyl-4-(R)-benzyl-2,5-imidazolidinedione-N-[1-(3-(5-(3-trifluoromethylbenzyl)-1,2,4-oxadiazolyl]carbonyl)-2-(S)-methylpropyl]acetamide among others. Methods of making these compounds and derivatives thereof are well known in the art and can be found, for example, in U.S. Pat. Nos. 5,807,829; 5,891,852; 5,869,455; 5,861,380; and 5,801,148, all of which are incorporated herein by reference in their entirety.

Other synthetic compounds of use include phenylenedialkanoate esters, which are effective in the mouse model. Specific examples of certain phenylenedialkanoate esters include, but are not limited to, 2,2′-(1,4-phenylene)dibutyric acid; tert-butyl-3-chloro-pivaloate; dimethyl-2,2′-(1,4-phenylene)diisobutyrate; 2,2′-(1,4-phenylene)diisobutyric acid; bis(sulfoxides); Obis(sulfones); and bis(4-(2′-carboxy-2′-methylpropylsulfonyl)phenyl)2,2′-(1,4-phenylene)diisobutyrate among others. More specifically, U.S. Pat. No. 5,216,022 teaches other compounds of potential use including: Benzyloxycarbonyl-L-valyl-N-[1-(2-[5-(3-methylbenzyl)-1,3,4-oxadiazolyl]carbonyl)-2-(S)-methylpropyl]-L-prolinamide (also known as CE-2072), Benzyloxycarbonyl-L-valyl-N-[1-(2-(3-methylbenzyl)-1,3,4-oxadiazolyl]carbonyl)-2-(S)-methylpropyl]-L-prolinamide; Benzyloxycarbonyl-L-valyl-N-[1-(2-(5-(methyl)-1,3,4-oxadiazoly]carbonyl)-2-(S)-methylpropyl]-L-prolinamide; Benzyloxycarbonyl)-L-valyl-N-[1-(2-(5-(3-trifluoromethylbenzyl)-1,3,4-oxadiazolyl]carbonyl)-2-(S)-methylpropyl]-L-prolinamide; (Benzyloxycarbonyl)-L-valyl-N-[1-(2-(5-(4-Dimethylaminobenzyl)-1,3,4-oxadiazolyl]carbonyl)-2-(S)-methylpropyl]-L-prolinamide; Benzyloxycarbonyl)-L-valyl-N-[1-(2-(5-(1-napthylenyl)-1,3,4-oxadiazolyl]carbonyl)-2-(S)-methylpropyl]-L-prolinamide; (Benzyloxycarbonyl)-L-valyl-[1-(3-(5-(3,4-methylenedioxybenzyl)-1,2,4-oxadiazolyl]carbonyl)-2-(S)-methylpropyl]-L-prolinamide; Benzyloxycarbonyl)-L-valyl-N-[1-(3-(5-(3,5-dimethylbenzyl)-1,2,4-oxadiazolyl]carbonyl)-2-(S)-methylpropyl]-L-prolinamide; (Benzyloxycarbonyl)-L-valyl-N-[1-(3-(5-(3,5-dimethoxybenzyl)-1,2,4-oxadiazolyl]carbonyl)-2-(S)-methylpropyl]-L-prolinamide; (Benzyloxycarbonyl)-L-valyl-N-[1-(3-(5-(3,5-ditrifluoromethylbenzyl)-1,2,4-oxadiazolyl]carbonyl)-2-(S)-methylpropyl]-L-prolinamide; (Benzyloxycarbonyl)-L-valyl-N-[1-(3-(5-(3-methylbenzyl)-1,2,4-oxadiazolyl]carbonyl)-2-(S)-methylpropyl]-L-prolinamide; (Benzyloxycarbonyl)-L-valyl-N-[1-(3-(5-(biphenylmethine)-1,2,4-oxadiazolyl]carbonyl)-2-(S)-methylpropyl]-L-prolinamide; (Benzylcarbonyl)-L-valyl-N-[1-(3-(5-(4-phenylbenzyl)-1,2,4-oxadiazolyl]carbonyl)-2-(S)-methylpropyl]-L-prolinamide; (Benzyloxycarbonyl)-L-valyl-N-[1-(3-(5-(3-phenylbenzyl)-1,2,4-oxadiazolyl]carbonyl)-2-(S)-methylpropyl]-L-prolinamide; (Benzyloxycarbonyl)-L-valyl-N-[1-(3-(5-(3-phenoxybenzyl)-1,2,4-oxadiazolyl]carbonyl)-2-(S)-methylpropyl]-L-prolinamide; (Benzyloxycarbonyl)-L-valyl-N-[1-(3-(5-(cyclohexylmethylene)-1,2,4-oxadiazolyl]carbonyl)-2-(S)-methylpropyl]-L-prolinamide; (Benzyloxycarbonyl)-L-valyl-N-[1-(3-(5-(3-trifluoromethyldimethylmethylene)-1,2,4-oxadiazolyl]carbonyl)-2-(S)-methylpropyl]-L-prolinamide; (Benzyloxycarbonyl)-L-valyl-N-[1-(3-(5-(1-napthylmethylene)-1,2,4-oxadiazolyl]carbonyl)-2-(S)-methylpropyl]-L-prolinamide; (Benzyloxycarbonyl)-L-valyl-N-[1-(3-(5-(3-pyridylmethyl)-1,2,4-oxadiazolyl]carbonyl)-2-(S)-methylpropyl]-L-prolinamide, (Benzyloxycarbonyl)-L-valyl-N-[1-(3-(5-(3,5-diphenylbenzyl)-1,2,4-oxadiazolyl]carbonyl)-2-(S)-methylpropyl]-L-prolinamide; (Benzyloxycarbonyl)-L-valyl-N-[1-(3-(5-(4-dimethylaminobenzyl)-1,2,4-oxadiazolyl]carbonyl)-2-(S)-methylpropyl]-L-prolinamide; 2-(5-[(Benzyloxycarbonyl)amino]-6-oxo-2-(4-fluorophenyl)-1,6-dihydro-1-pyrimidinyl]-N-[1-(3-(5-(3-trifluoromethylbenzyl)-) 1,2,4-oxadiazolyl]carbonyl)-(S)-2-methylpropyl]acetamide; 2-(5-Amino-6-oxo-2-(4-fluorophenyl)-1,6-dihydro-1-pyrimidinyl]-N-[1-(3-(5-(3-trifluoromethylbenzyl)-1,2,4-oxadiazolyl]carbonyl)-2-(S)-methylpropyl]acetamide; 2-(5-[(Benzyloxycarbonyl)amino]-6-oxo-2-(4-fluorophenyl)-1,6-dihydro-1-pyrimidinyl]-N-[1-(2-(5-(3-methylbenzyl)-1,3,4-oxadiazolyl]carbonyl)-(S)-2-methylpropyl]acetamide; 2-(5-Amino-6-oxo-2-(4-fluorophenyl)-1,6-dihydro-1-pyrimidinyl]-N-[1-(2-(5-(3-methylbenzyl)-1,3,4-oxadiazolyl]carbonyl)-2-methylpropyl]acetamide; (Pyrrole-2-carbonyl)-N-(benzyl)glycyl-N-[1-(2-(5-(3-methylbenzyl)-1,3,4-oxadiazolyl]carbonyl)-2-(S)-methylpropyl]amide; (Pyrrole-2-carbonyl)-N-(benzyl)glycyl-N-[1-(3-(5-(3-trifluoromethylbenzyl)]-1,2,4-oxadiazolyl)-(S)-methylpropyl]amide; (2S,5S)-5-Amino-1,2,4,5,6,7-hexahydroazepino-[3,2,1]-indole-4-one-carbonyl-N-[1-(2-(5-(3-methylbenzyl)-1,3,4-oxadiazolyl]carbonyl)-(R,S)-2-methylpropyl]amide; BTD-[1-(2-(5-(3-methylbenzyl)-1,3,4-oxadiazolyl]carbonyl)-2-(S)-methylpropyl]amide; (R,S)-3-Amino-2-oxo-5-phenyl-1,4,-benzodiazepine-N-[1-(2-(5-(3-methylbenzyl)-1,3,4-oxadiazolyl]carbonyl)-2-(S)-methylpropyl]acetamide; (Benzyloxycarbonyl)-L-valyl-2-L-(2,3-dihydro-1H-indole)-N-[1-(2-(5-(3-methylbenzyl)-1,3,4-oxadiazolyl]carbonyl)-2-(S)-methylpropyl]amide; (Benzyloxycarbonyl)-L-valyl-2-L-(2,3-dihydro-1H-indole)-N-[1-(3-(5-(3-trifluoromethylbenzyl)-1,2,4-oxadiazolyl]carbonyl)-2-(S)-methylpropyl]amide; Acetyl-2-L-(2,3-dihydro-1H-indole)-N-[1-(2-(5-(3-methylbenzyl)-1,3,4-oxadiazolyl]carbonyl)-2-(S)-methylpropyl]amide; 3-(S)-(Benzyloxycarbonyl)amino)-F-lactam-N-[1-(2-(5-(3-methylbenzyl)-1,3,4-oxadiazolyl]carbonyl)-2-(S)-methylpropyl]acetamide; 3-(S)-(Amino)-F—lactam-N-[1-(2-(5-(3-methylbenzyl)-1,3,4-oxadiazolyl]carbonyl)-2-(S)-methylpropyl]acetamide trifluoroacetic acid salt; 3-(S)-[(4-morpholinocarbonyl-butanoyl)amino]-ε-lactam-N-[1-(2-(5-(3-methylbenzyl)-1,3,4-oxadiazolyl]carbonyl)-2-(R,S)-methylpropyl]acetamide; 6-[4-Fluorophenyl]-ε-lactam-N-[1-(2-(5-(3-methylbenzyl)-1,3,4-oxadiazolyl]carbonyl)-2-(S)-methylpropyl]acetamide; 2-(2-(R,S)-Phenyl-4-oxothiazolidin-3-yl]-N-[1-(2-(5-(3-methylbenzyl)-1,3,4-oxadiazolyl]carbonyl)-2-(S)-methylpropyl]acetamide; 2-(2-(R,S)-phenyl-4-oxothiazolidin-3-yl]-N-[1-(2-(5-(3-methylbenzyl)-1,3,4-oxadiazolyl]hydroxymethyl)-2-(S)-methylpropyl]acetamide; 2-(2-(R,S)-Benzyl-4-oxothiazolidin-3-yl]-N-[1-(2-(5-(3-methylbenzyl)-1,3,4-oxadiazolyl]carbonyl)-2-(S)-methylpropyl]-acetamide; 2-(2-(R,S)-Benzyl-4-oxothiazolidin-3-yloxide]-N-[1-(3-(5-(3-trifluoromethylbenzyl)-1,2,4-oxadiazolyl]carbonyl)-2-(R,S,)-methylpropyl]acetamide; (1-Benzoyl-3,8-quinazolinedione)-N-[1-(2-(5-(3-methylbenzyl)-1,3,4-oxadiazolyl]carbonyl)-2-(S)-methylpropyl]acetamide; (1-Benzoyl-3,6-piperazinedione)-N-[1-(2-(5-(3-methylbenzyl)-1,3,4-oxadiazolyl]carbonyl)-2-(S)-methylpropyl]acetamide; (1-Phenyl-3,6-piperazinedione)-N-[1-(2-(5-(3-methylbenzyl)-1,3,4-oxadiazolyl]carbonyl)-2-(S)-methylpropyl]acetamide; [(1-Phenyl-3,6-piperazinedione)-N-[1-(3-(5-(3-trifluoromethylbenzyl)-1,2,4-oxadiazolyl]carbonyl)]-2-(S)-methylpropyl]acetamide; 3-[(Benzyloxycarbonyl)amino]-quinolin-2-one-N-[1-(2-(5-(3-methylbenzyl) 1,3,4-oxadiazolyl]carbonyl)-2-(S)—methylpropyl]acetamide; 3-[(Benzyloxycarbonyl)amino]-7-piperidinyl-quinolin-2-one-N-[1-(2-(5-(3-methylbenzyl)-1,3,4-oxadiazolyl]carbonyl)-2-(S)-methylpropyl]acetamide; 3-(Carbomethoxy-quinolin-2-one-N-[1-(2-(5-(3-methybenzyl)-1,3,4-oxadiazolyl]carbonyl)-2-(S)-methylpropyl]acetamide; 3-(Amino-quinolin-2-one)-N-[1-(2-(5-(3-methylbenzyl)-1,3,4-oxadiazolyl]carbonyl)-2-(S)-methylpropyl]acetamide; 3-[(4-Morpholino)aceto]amino-quinolin-2-one-N-[1-(2-(5-(3-methylbenzyl)-1,3,4-oxadiazolyl]carbonyl)-2-(S)-methylpropyl]acetamide; 3,4-Dihydro-quinolin-2-one-N-[1-(2-(5-(3-methylbenzyl)-1,3,4-oxadiazolyl]carbonyl)-2-(S)-methylpropyl]acetamide; 1-Acetyl-3-(4-fluorobenzylidene) piperazine-2,5-dione-N-[1-(2-(5-(3-methylbenzyl)-1,3,4-oxadiazolyl]carbonyl)-2-(S)-methylpropyl]acetamide; 1-Acetyl-3-(4-dimethylaminobenzylidene)piperazine-2,5-dione-N-[1-(2-(5-(3-methylbenzyl)-1,3,4-oxadiazolyl]carbonyl)-2-(S)-methylpropyl]acetamide; 1-Acetyl-3-(4-carbomethoxybenzylidene)piperazine-2,5-dione-N-[1-(2-(5-(3-methylbenzyl)-1,3,4-oxadiazolyl]carbonyl)-2-(S)-methylpropyl]acetamide; 1-Acetyl-3-[(4-pyridyl)methylene]piperazine-2,5-dione-N-[1-(2-(5-(3-methylbenzyl)-1,3,4-oxadiazolyl]carbonyl)-2-(S)-methylpropyl]acetamide; 4-[1-Benzyl-3-(R)-benzyl-piperazine-2,5,-dione]-N-[1-(2-[5-(3-methylbenzyl)-1,3,4-oxadiazolyl]carbonyl)-2-(S)-methylpropyl]acetamide; 4-[1-Benzyl-3-(S)-benzyl piperazine-2,5,-dione]-N-[1-(2-(5-(3-methylbenzyl)-1,3,4-oxadiazolyl]carbonyl)-2-(S)-methylpropyl]acetamide; 4-[1-Benzyl-3(R)-benzylpiperazine-2,5,-dione]-N-[1-(3-(5-(3-trifluoromethylbenzyl)-1,2,4-oxadiazolyl]carbonyl)-2-(S)-methylpropyl]acetamide; 4-[1-Benzyl-3-(S)-benzylpiperazine-2,5,-dione]-N-[1-(3-(5-(3-trifluoromethylbenzyl)-1,2,4-oxadiazolyl]carbonyl)-2-(S)-methylpropyl]acetamide; 4-[1-Benzyl-3-(S)-benzyl piperazine-2,5,-dione]-N-[1-(3-(5-(2-dimethylaminoethyl)-1,2,4-oxadiazolyl]carbonyl)-2-(S)-methylpropyl]acetamide; 4-[1-Methyl-3-(R,S)-phenylpiperazine-2,5,-dione]-N-[1-(3-(5-(3-trifluoromethylbenzyl)-1,2,4-oxadiazolyl]carbonyl)-2-(S)-methylpropyl]acetamide; 4-[1-Methyl-3-(R,S)-phenylpiperazine-2,5,-dione]-N-[1-(2-(5-(3-methylbenzyl)-1,3,4-oxadiazolyl]carbonyl)-2-(S)-methylpropyl]acetamide; 4-[1-(4-Morpholinoethyl)3-(R)-benzylpiperazine-2,5,-dione]-N-[1-(2-(5-(3-methylbenzyl)-1,3,4-oxadiazolyl]carbonyl)-2-(S)-methylpropyl]acetamide; 5-(R,S)-Phenyl-2,4-imidazolidinedione-N-[1-(2-(5-(3-methylbenzyl)-1,3,4-oxadiazolyl]carbonyl)-2-(S)-methylpropyl]acetamide; 5-(R)-Benzyl-2,4-imidazolidinedione-N-[1-(2-(5-(3-methylbenzyl)-1,3,4-oxadiazolyl]carbonyl)-2-(S)-methylpropyl]acetamide; 5-(S)-Benzyl-2,4-imidazolidinedione-N-[1-(2-(5-(3-methylbenzyl)-1,3,4-oxadiazolyl]carbonyl)-2-(S)-methylpropyl]acetamide; 5-(S)-Benzyl-2,4-imidazolidinedione-N-[1-(3-(5-(3-trifluoromethylbenzyl)-1,2,4-oxadiazolyl]carbonyl)-2-(S)-methylpropyl]acetamide; 5-(R)-Benzyl-2,4-imidazolidinedione-N-[1-(3-(5-(3-trifluoromethylbenzyl)-1,2,4-oxadiazolyl]carbonyl)-2-(S)-methylpropyl]acetamide; 1-Benzyl-4-(R)-benzyl-2,5-imidazolidinedione-N-[1-(2-(5-(3-methylbenzyl)-1,3,4-oxadiazolyl]carbonyl)-2-(S)-methylpropyl]acetamide; and 1-Benzyl-4-(R)-benzyl-2,5-imidazolidinedione-N-[1-(3-(5-(3-trifluoromethylbenzyl)-1,2,4-oxadiazolyl]carbonyl)-2-(S)-methylpropyl]acetamide among others. Methods of making these molecules and derivatives thereof are well known in the art and can be found in aforementioned U.S. Pat. No. 5,216,022, which is incorporated herein by reference in its entirety.

Other compounds that can be used include those disclosed in PCT Publication Nos. WO 98/20034; WO98/23565, which discusses aminoguanidine and alkoxyguanidine compounds; WO98/50342, which discusses bis-aminomethylcarbonyl compounds; WO98/50420, which discusses cyclic and other amino acid derivatives; WO 97/21690, which discusses D-amino acid containing derivatives; WO 97/10231, which discusses ketomethylene group-containing compounds; WO 97/03679, which discusses phosphorous containing compounds; WO 98/21186, which discusses benzothiazo and related heterocyclic compounds; WO 98/22619, which discusses a combination of compounds; WO 98/22098; WO 97/48706, which discusses pyrrolo-pyrazine-diones; WO 97/33996, which discusses human placental bikunin (recombinant); WO 98/46597, which discusses complex amino acid containing molecule.

Other useful compounds of the invention include, but are not limited to, tetrazole derivatives such as those disclosed in PCT Publication No. WO 97/24339; guanidinobenzoic acid derivatives as disclosed in PCT Publication No. WO 97/37969 and U.S. Pat. Nos. 4,283,418; 4,843,094; 4,310,533; 4,283,418; 4,224,342; 4,021,472; 5,376,655; 5,247,084; and 5,077,428; phenylsulfonylamide derivatives such as those discussed in PCT Publication No. WO 97/45402; sulfide, sulfoxide and sulfone derivatives such as those discussed in PCT Publication No. WO 97/49679; amidino derivatives such as those discussed in PCT Publication No. WO 99/41231; amidinophenol derivatives such as those discussed in U.S. Pat. Nos. 5,432,178; 5,622,984; 5,614,555; 5,514,713; 5,110,602; 5,004,612; and 4,889,723 among many others.

In general compounds exhibiting actin modulating activity, in particular actin binding or actin polymerization inhibiting activity, such as AAT, peptides derived from AAT, or peptides that are analogous or homologous to C-terminal end of AAT, and synthetic compounds that modulate actin display HIV-1-suppressive effects in vitro and in vivo.

### Example 14

This example shows synergy of intracellular actin binding related molecules with anti-HIV-1 drugs.

In some embodiments of the invention, a combination therapy with other anti-HIV-1 drugs are contemplated. Among these compositions are nucleoside reverse transcriptase (RT) inhibitors such as Retrovir (AZT/zidovudine; Glaxo Wellcome); Epivir (3TC, lamivudine; Glaxo Wellcome); Videx (ddI/didanosine; Bristol-Myers Squibb); Hivid (ddC/zalcitabine; Hoffmann-La Roche); Zerit (d4T/stavudine; Bristol-Myers Squibb); Ziagen (abacavir, 1592U89; Glaxo Wellcome); Hydrea (Hydroxyurea/HO; Bristol-Myers Squibb) and non-nucleoside reverse transcriptase inhibitors (NNRTIs) such as Viramune (nevirapine; Roxane Laboratories); Rescriptor (delavirdine; Pharmacia & Upjohn); Sustiva (efavirenz, DMP-266; DuPont Merck); Preveon (adefovir dipivoxil, bis-POM PMEA; Gilead).

Also useful combination therapy compounds include aspartyl protease inhibitors (PI's) including Fortovase (saquinavir; Hoffmann-La Roche); Norvir (ritonavir; Abbott Laboratories); Crixivan (indinavir; Merck & Company); Viracept (nelfinavir; Agouron Pharmaceuticals); Tenofovir (Viread, Gilead); Tipranovir (PNU-140690, Pharmacia & Upjohn); Atazanavir (Reyataz, Bristol-Myers Squibb Company); Fuzeon (Enfuvirtide, Roche); and Agenerase (amprenavir/141W94; Glaxo Wellcome).

These exemplary methods illustrate HIV-1-suppressive activity in all three in vitro models; U1 cells, PBMC, and MAGI cells. To anyone skilled in the art it is obvious that these models closely relate to the in vivo situation. This is further supported by the commercial and clinical success of existing, publicly available anti-HIV-1 drugs which were all initially tested in similar in vitro models. The results from such models are highly predictive of success or failure in clinical settings. Experiments conducted in U1 cells establish the blockade of HIV-1 production in a chronic infection model. This inhibitory effect was observed for all stimuli tested, including inflammatory cytokines (IL-18, IL-6, TNF) LPS and hyperosmolarity. The inhibitory effect was potent with a range of inhibition of 73-100%.

Results obtained in HIV-1-infected PBMC demonstrate several characteristics of AAT inhibition. Experiments were performed in PBMC from three donors infected in the absence or presence of AAT during infection. AAT effects during the infection period were established by the enhancement of AAT effect when added to PBMC following infection and cultured for 4 days. Enhancement of 4 day AAT effect was manifested by a larger maximal suppression and by suppression at lower AAT concentrations. Maximal p24 reductions in PBMC exposed to AAT for 4 days were 46% and 71% for cells infected in the absence or presence of AAT, respectively. For cells infected in the absence of AAT, a significant suppressive effect was observed for post-infection when AAT was added at 5 and 4 mg/ml. For cells infected in the presence of AAT, a significant effect was obtained when AAT was added at 5, 4, 3, and 2 mg/ml. Considered together, these data indicate a reversible enhancing effect of AAT when present at the time of PBMC infection.

Experiments performed in MAGI-CCR-5 cells indicated inhibitory effects of AAT and related compounds on early infection-associated events. The observed dose-dependent effect was maximal at 5 mg/ml AAT, where 76% inhibition was observed compared to control (HIV-1 added in the absence of AAT). These results indicate that AAT inhibits HIV-1 events prior to integration into the host-cell genome (cell-surface receptor binding, internalization, integration, uncoating, reverse transcription, translation and protein processing and tat activation).

Also, peptides analogous or homologous to the C-terminal end of AAT, and other actin modulating compounds displayed HIV-1-suppressive effect during both early (PBMC and MAGI-CCR-5 cell results) and late (U1 cell results) events associated with HIV-1 infection. Unexpectedly, the synergy appears to exist between known AIDS drugs belonging to RT and PI classes and actin modulators.

### Example 15

This example shows that AAT prolongs islet allograft survival.

Islets isolated from DBA/2 mice (H-2d) were transplanted under the left renal capsule of streptozotocin (STZ-) induced hyperglycemic C57BL/6 mice (H-2b). Blood glucose was measured three times a week. As expected, untreated recipient mice exhibited glucose levels above 300 mg/dl on day 10 and later, reflecting the occurrence of graft failure. Human AAT or albumin was injected intraperitoneally (i.p.) one day before transplantation and every 3 days thereafter. Treatment with human albumin (6 mg) resulted in graft rejection comparable to that of untreated recipient mice. In contrast, recipient mice that received AAT (2 mg) exhibited prolonged graft function. Neither of the partial treatment protocols, i.e., days −1, 0 and 1 (‘early treatment’) or days 2 and beyond (‘late treatment’) prolonged allograft survival.

AAT-treated mice developed anti-human-AAT antibodies. Individual mice exhibited anti-human-AAT antibodies at various time points. To ascertain that the antibodies reduce the protective effect of AAT, a group of mice was pre-exposed (immunized) to human AAT two months before being rendered hyperglycemic and transplanted with allogeneic islets. These graft recipients were treated with the full AAT protocol, despite exhibiting high titers of specific antibodies before engraftment, and displayed rapid graft rejection. Day 15 was chosen to depict an association between antibody formation and loss of AAT protective activity. At this time point AAT-treated mice were divided into positive and negative producers of anti-human-AAT antibodies. On day 15 all antibody-positive mice were hyperglycemic and all antibody-negative mice were normoglycemic.

### AAT Inhibits Cellular Infiltration.

To address the possibility that AAT affects immune cell infiltration, two models of cell emigration were examined: thioglycolate (ThG)-elicited peritoneal infiltration, and cellular infiltration due to intraperitoneal injection of MHC-incompatible fibroblasts. C57BL/6 mice were injected i.p. with 0.1 ml saline, human albumin, human AAT or oxidized AAT, prior to injection of 1 ml thioglycolate. Peritoneal lavage was performed 24 and 48 hours later and cells were counted and stained with specific antibodies for FACS analysis. There was a progressive increase in total cell count at 24 and 48 hours in mice injected with ThG, whereas no significant increase was observed in mice injected with AAT and ThG. At 48 hours, total cell count in peritoneal lavage of AAT-treated mice was 50% of that of control. Total cell count in mice that received albumin control was similar to that of saline-treated mice. There was a dose-dependent effect of AAT in that one-sixth the dose was found to reduce cell count to a lesser extent in a significant manner. Oxidized AAT did not affect cellular infiltrate at 1 mg.

Freshly harvested NIH-3T3 cells, derived from BALB/c mice (H-2d), were injected i.p. to C57BL/6 mice (H-2b) pretreated with i.p. saline or human AAT (2 mg). Lavage was performed on days 1 through 5. Introduction of allogeneic cells evoked a cellular infiltrate that consisted of early appearing neutrophils and activated macrophages, and late appearing CD3+ and NK cells. AAT-treated mice exhibited a reduction in neutrophils, CD3+ and NK cells.

To evaluate the level of cellular infiltration into grafted islets, grafts from AAT- and ALB-treated recipient mice were removed on day 7, fixed in paraformaldehyde and stained with Hematoxilin and Eosin. A cellular infiltrate was demonstrable regardless of AAT treatment, and included neutrophils and lymphocytes. However, the infiltrates evoked by grafts of ALB-treated recipient mice were more massive and caused the disruption of islet borders, compared to intact islets of AAT-treated recipient mice. To evaluate islet function, grafts from AAT- and ALB-treated recipient mice were removed on day 15, and immunohistochemistry was performed with anti-insulin antibodies. Insulin production was preserved on day 15 in islets of AAT-treated-recipients.

AAT Modifies Islet Response to Proinflammatory Mediators.

Various islet responses to IL-1β/IFNγ were examined in vitro. Islets exposed to IL-1β/IFNγ for 72 hours produced nitric oxide (NO) in a concentration-dependent manner and exhibit NO-dependent loss of viability. In the presence of AAT, less NO was produced and greater islet viability was obtained. The production of MIP-1α was decreased in the presence of AAT, particularly when stimulated by low concentrations of IL-1β/IFNγ. Notably, TNFα level in supernatants was markedly diminished by AAT. Insulin induction was inhibited by IL-1β/IFNγ but was intact in the presence of IL-1β/IFNγ plus AAT.

To test the effect of AAT on islets in vivo, STZ toxicity was evaluated. AAT (2 mg) was administered one day before, on the same day and a day after STZ injection. Immunohistochemistry of pancreata with anti-insulin antibodies at 48 hours after STZ injection revealed more insulin-producing cells in islets of AAT- than ALB-treated mice (26.3%±2.6 and 12.8%±2.3 insulin-producing cells per islet, respectively). Immune cell content of freshly isolated islets was evaluated by FACS analysis. Islets contain CD45+ cells that are also positive for the monocytic/granulocytic markers GR1 and F4/80. This cell population responded to AAT with decreased surface MHC class II.

### AAT Inhibits Release of Membrane TNFα

Proteolytic cleavage of membrane TNFα releases soluble TNFα from activated cells by the action of TNFα-converting-enzyme (TACE). The levels of membrane TNFα on stimulated islets in the presence of AAT was examined. The effect of AAT was then compared to that of a TACE inhibitor. Both AAT and TACE inhibitor decreased TNFα levels in supernatants of islets exposed to IL-1β/IFNγ. Under these conditions, membrane TNFα accumulated on the cell surface of CD45+ islet cells.

To assess the possibility that islet protection occurred via inhibition of release of membrane TNFα in vivo, TACE inhibitor, p75 TNFα receptor or AAT were introduced to mice prior to STZ injection. Although all mice developed hyperglycemia after day 4, the progression of β-cell toxicity was significantly affected by treatments. The effect of STZ at 48 hours was decreased in the presence of AAT (a decrease of 23.2%±2.3 in fasting glucose levels compared to STZ/saline injected mice). The effect of TACE inhibitor and p75 TNFα receptor was not as profound. Similarly, TACE inhibitor prolonged islet graft survival to a lesser extent than AAT.

Splenocytes that were harvested 48 hours after ThG injection produced TNFα in culture. AAT administered prior to thioglycolate decreased TNFα release from cultured splenocytes. A similar trend was found with IFNγ, signifying that the response to ThG had implications beyond the peritoneal compartment and that pretreatment with AAT was able to diminish it.

### Example 16

The effects of AAT on the actin cytoskeleton of three different cell types, including Hela cells, primary human fibroblast cells, and a murine macrophage cell line, RAW264.7 were analyzed (data not shown). After incubating the cells with AAT for 16 hours, the cells were permeabilized and stained with phalloidin. The actin cytoskeleton stained red and the nucleus (stained with another fluorescent dye, DAPI) appeared blue (data not shown). Control cells had a characteristic phalloidin staining of the actin cytoskeleton, with a single nucleus enclosed within a large, well-defined cytoplasmic region. Effects on cellular structure can be seen at 5 mg/mL of AAT with the appearance of large, multinucleated cells (syncitium), which may indicate either inhibition of cell separation during cellular mitosis or active fusion of single cells. Furthermore, those cells containing single nuclei had a cytoskeletal structure which was clearly contracted compared to Control cells. Similar contractions of the cytoskeleton of single-nucleated cells at 2 mg/mL of AAT, although fewer syncitia, were noted (data not shown). At 7 mg/mL, larger syncitia and an increased frequency of cells with collapsed cytoskeletons were evident.

These primary cells spread on a polystyrene surface with greater surface areas than those of Hela cells, which allowed for enhanced visualization of intracellular actin fibers. Control fibroblasts have a highly defined cytoskeletal structure, with several filopodia and a single nucleus. When exposed to 5 mg/mL of AAT, the cytoplasmic area appeared to decrease in volume, but the cytoskeletal-actin fibers remained well defined and appeared to be unaffected. When fibroblasts were exposed to 7 mg/mL of AAT, the cytoplasmic area was further decreased and the filopodia disappeared. The brightness of the phalloidin staining in the affected cells appeared to indicate that a similar amount of actin was present in these cells compared to Control cells, but the actin was compacted into a smaller volume. These observations appeared to suggest actin rearrangements and alterations of cell morphology.

RAW264.7 cells, a murine macrophage cell line that adheres to polystyrene tissue culture wells, have large nuclei but relatively limited cytoplasm. When treated with 5 mg/mL of AAT, the cellular membranes were severely affected. Multiple filipodia extended into the extracellular space, and the cellular membrane had an indistinct or fuzzy appearance. In addition, the cytoplasmic space appeared larger than in Control cells, and the cytoskeleton was dysmorphic and unorganized. At 7 mg/mL, these effects became more pronounced and affected all of the cells observed.

AAT affected the actin cytoskeleton in each of these three different cell types. However, the effects were specific for each cell type. The Hela and fibroblast cells demonstrated AAT-associated loss of cytoskeletal structure and compaction of the cytoplasm. The RAW264.7 cells appeared to gain cytoplasmic space, but the cytoskeletal actin fibers appeared disorganized. Despite the different specific effects observed in response to incubation with AAT, each cell type displayed significant deformation of cellular morphology and an altered actin cytoskeleton.

Two forms of actin exist intracellularly: monomeric G (globular)-actin and multimeric F (filamentous)-actin. Polymerization of G-actin monomers produces F-actin fibers that form actin filaments. Networks of F-actin filaments comprise the cytoskeleton that confers cellular structure. The actin cytoskeletal structure is not only needed for cellular processes such as receptor dimerization and cell signaling, but also allows directed movement of cells (cytokinesis). The actin cytoskeleton undergoes constant rearrangement during cell movement, with actin monomers added to the growing (plus) end and removed from the minus end to permit extensions and contractions of cytoplasmic processes.

An actin sedimentation assay was used to determine whether AAT binds to G or F-actin. This assay is based on the observation that F-actin is able to form large molecular-weight filaments and structures. Due to differences in mass, F-actin and G-actin can be separated using high-speed centrifugation. In these experiments, G-actin and AAT were incubated under conditions which induced polymerization of G-actin into F-actin. Upon formation of F-actin, actin-binding proteins bind the F-actin and co-sediment following centrifugation, leaving G-actin binding proteins or non-actin binding proteins in the supernatant. Pelleted proteins and supernatants were electorphoresed into an SDS-PAGE gel and the resulting bands imaged quantitatively to determine the relative amounts of sedimented and unsedimented proteins. Proteins that sediment bind actin, whereas proteins that do not appear in the pellet do not bind actin.

### Example 17

This Example Shows that AAT Binds to U1 Cells

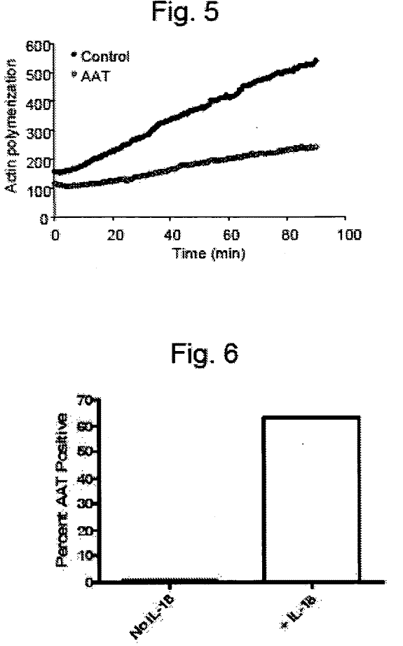

The ability of AAT to bind to the surface of different types of cells was examined. By identifying the cell types that bind AAT, directed experiments that explain how AAT inhibits HIV were designed. U1 cells can be activated to induce HIV-1 replication using pro-inflammatory stimuli. Flow cytometry was used to assess AAT binding to the surface of these cells. U1 cells were stimulated using 5 nM IL-18 overnight. The cells were then washed and incubated with 3 mg/mL of AAT for 1 hr at 37° C. Unstimulated U1 cells incubated with 3 mg/mL AAT were used as a control. After incubation with AAT to allow binding to cell surface receptors, the cells were washed and incubated with a goat anti-human AAT antibody conjugated to FITC for 30 min. As shown in FIG. 6, flow cytometric analysis revealed that less than 1% of unstimulated U1 cells (no IL-18 added) were bound to AAT. However, when the cells were stimulated with IL-18, 63% of all U1 cells became bound to AAT. Analyses showed that the cells that were bound to AAT were cells that had been activated.

### Example 18

This example shows that AAT binds to PBMC.

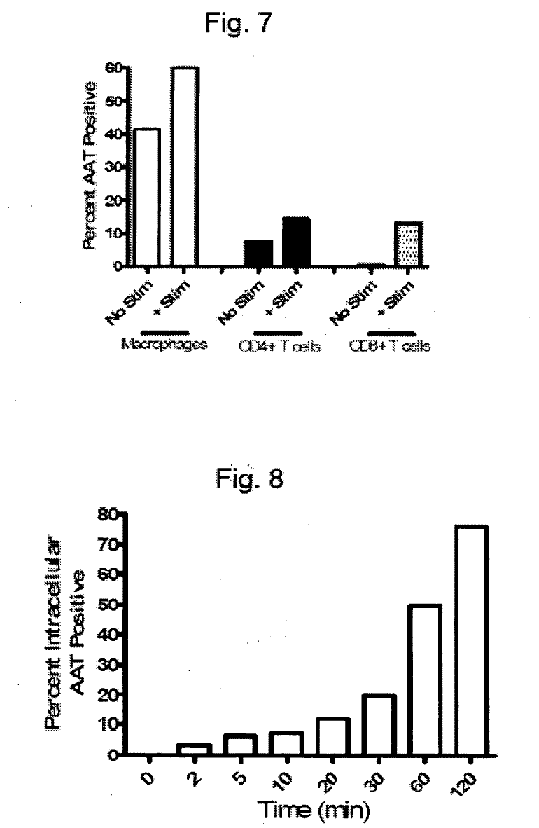

Experiments to determine which cells in peripheral blood mononuclear cells (PBMC) bind to AAT were performed. PBMC were isolated from a healthy volunteer and stimulated with phytohemaglutinin (PHA) and IL-2 for 3 days (+Stim). As a control, freshly isolated, but unstimulated PBMC were also analyzed (No Stim). The PBMC were washed and incubated with 3 mg/mL of AAT for 1 hr at 37° C. The PBMC were then washed and incubated with antibodies directed against AAT, CD4 (Helper T cells), CD8 (Cytotoxic T cells) or CD14 (macrophages). As shown in FIG. 7, 40% of unstimulated monocytes were bound to AAT (substantially more than resting U1 cells). Upon stimulation, approximately 60% of monocytes were bound to AAT. A smaller percentage of T cells (both CD4 and CD8) were bound to AAT. However, similar to macrophages, stimulation with PHA and IL-2 increased the number of cells that bind to AAT.

### Example 19

This example shows cellular fate of bound AAT.

The binding of AAT to the outside of a cell to alter the actin cytoskeleton inside a cell was investigated. In an attempt to determine if AAT is able to enter the cell (and thereby interact directly with intracellular actin), flow cytometry was used to quantitate the number of cells containing intracellular (endocytosed) AAT. HIV-1-infected U1 cells were stimulated overnight with phorbol 12-myristate 13-acetate (PMA), washed, and incubated with 5 mg/mL AAT. At various time points following addition of AAT, cell aliquots were removed, fixed, permeabilized and incubated with a FITC-labeled anti-AAT antibody. Permeabilization enabled staining of intracellular AAT. As shown in FIG. 8, up to 75% of the activated U1 cells contained intracellular AAT after 120 minutes of incubation. This indicates that after AAT binds to the cell surface of activated cells, it is endocytosed into the interior of the cell where it is believed to initiate events that alter the actin cytoskeleton.

### Example 20

This example shows role of serine protease inhibition activity of AAT in blocking HIV-1 production.

The anti-serine protease function of AAT was examined for its role in the inhibitory effects on HIV-1. AAT was modified to reduce its serine protease inhibitory function to undetectable levels. Quantitation of the anti-protease activity of AAT was determined using an elastase neutralization assay. In this assay porcine elastase was incubated with a cleavable substrate. Upon cleavage of the substrate by elastase, a color-generating product was released, which was quantitated using a spectrophotometer. Addition of fresh AAT to this reaction almost completely inhibited the cleavage of the substrate by elastase (data not shown). Using this method, the anti-protease function of AAT was determined to have been reduced to undetectable levels. The inactivated AAT was then added to U1 cells, which was then stimulated with IL-18 and compared to native AAT. Inactivated AAT had an almost identical inhibitory profile to that of native AAT, with complete suppression of HIV-1 production at 4 mg/mL of AAT, data not shown. These data indicate that the suppressive effect of AAT on HIV-1 production does not require AAT serine protease inhibitor activity.

### Example 21

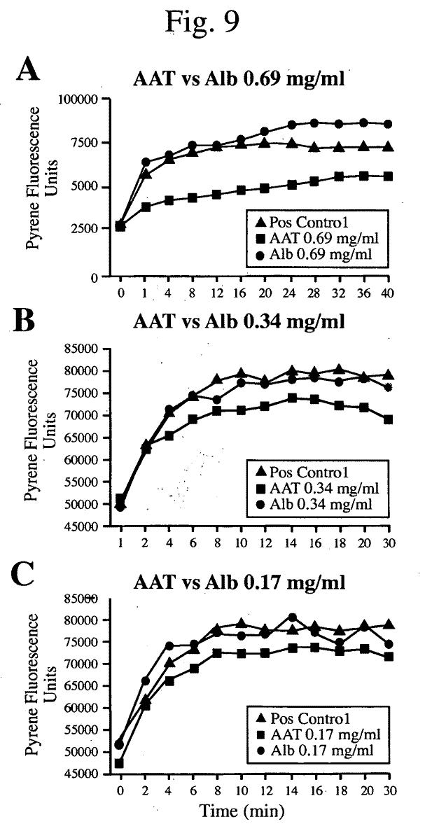

An actin polymerization buffer was added to pyrenated G-actin (control), and the formation of F-actin was followed over a period of 60 minutes. As shown in FIG. 9A-9C, AAT concentrations of 0.69 mg/ml (FIG. 9A), 0.34 mg/ml (FIG. 9B), and 0.17 mg/ml (FIG. 9C) were used. Significant inhibition of F-actin filament formation at 0.69 and 0.34 mg/ml of AAT was observed. Similar concentrations of human albumin (Alb) were used as controls and demonstrated no inhibition of actin polymerization. These experiments show that AAT interacts with actin and inhibits actin polymerization

In separate experiments (data not shown), F-actin filaments were produced and AAT was added to determine if AAT had depolymerization properties. As a control, Getsolin™, a protein that has known depolymerization properties, was added to F-actin filaments. While gelsolin was found to actively dissociate, i.e., depolymerize, F-actin polymers, AAT had no effect on F-actin indicating that AAT prevents polymerization of actin monomers but has no depolymerization activity.

### Example 22

The ability of AAT and actin to interact with each other and form aggregates of higher molecular weight protein complexes were examined. AAT and actin monomers or 1 micron actin filaments were incubated and electrophoresed on a Native protein gel. Native protein gels separate protein complexes based on size (molecular weight), but because they do not contain a detergent such as SDS (as in SDS-PAGE), the protein complexes are not denatured and migrate in their aggregated forms. Thus, if AAT interacts and binds with actin to form a higher molecular weight structure, individual (monomeric) AAT and actin, and higher molecular weight complexes consisting of AAT and actin were expected.

In this experiment, two forms of actin were used in the binding experiment: monomeric actin (mono actin) and filamentous actin that was prepared such that the filaments were approximately 1 micron in length (1 micron actin). AAT and actin were mixed at different mass ratios and incubated at 37° C. for 2 hr. The AAT:actin mass ratios used were 1:1, 2:1, 4:1, 1:2 and 1:4. The proteins were then electrophoresed into a Native gel, transferred to PVDF membrane, and a Western blot was performed and probed for AAT and actin proteins. Expected molecular weights were 52 kDa for AAT, 43 kDa for actin, and higher molecular weight aggregates consisting of AAT and actin.

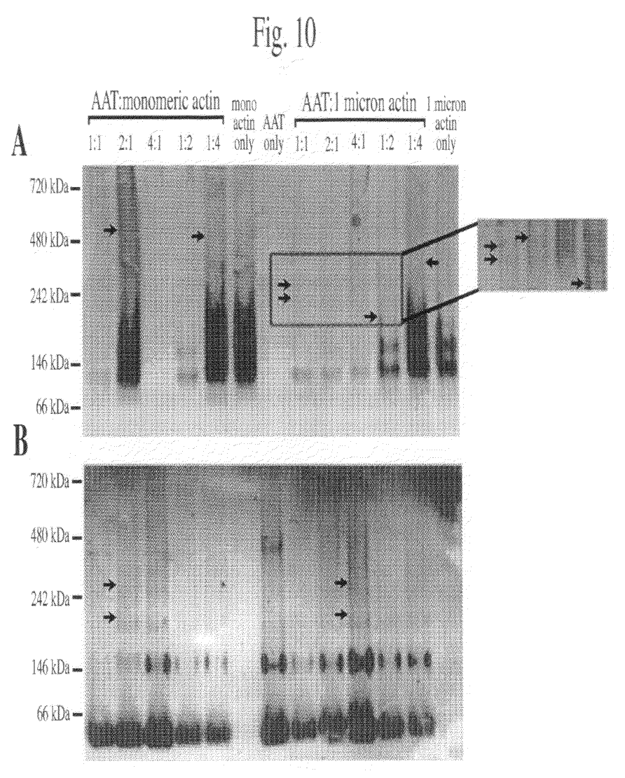

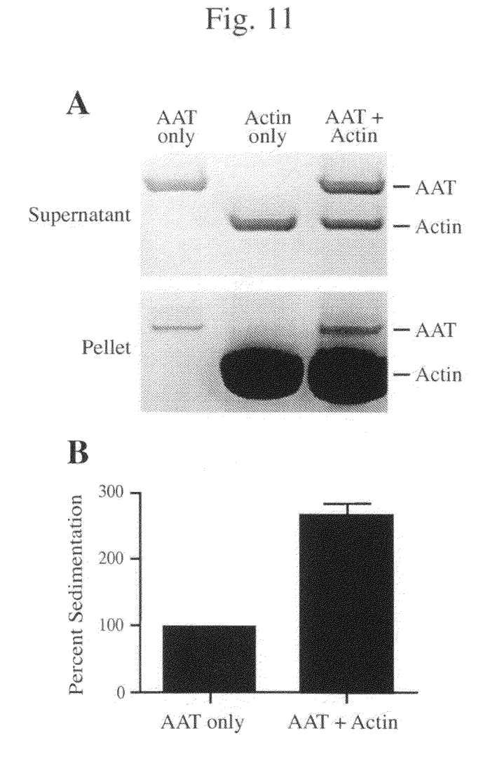

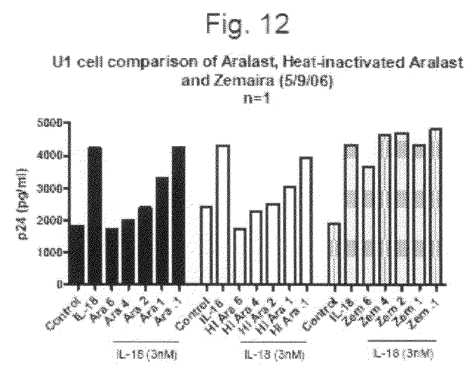

As shown in FIG. 10, a Western blot of the proteins probed with either actin (FIG. 10A) or AAT (FIG. 10B) antibodies resulted in several positive larger molecular weight protein bands. In FIG. 10, a region of the blot was overexposed in order to detail faint bands present in those lanes. In the Western blot probed with an actin antibody (FIG. 10A), two unique higher molecular weight complexes were visualized at approximately 500 kDa in the AAT/monomeric actin mixtures containing a 2:1 actin are 52 kDa and 43 kDa proteins respectively (a total of 95 kDa), it is probable that multimeric complexes are forming with several actin molecules binding to AAT or vice-versa. Lower molecular weight complexes could not be sufficiently visualized because of high background. When examining actin/AAT complexing using 1 micron actin filaments (right side of FIG. 10A), more bands were visualized, including bands at 250 kDa and 280 kDa in the 1:1 molar ratio lane (lane 8), a 180 kDa band in the 1:2 molar ratio (lane 11), and a 250 kDa band in the 1:4 molar ratio lane (lane 9).

To confirm the presence of both actin and AAT in these complexes, an identical blot was probed with an anti-AAT antibody (FIG. 10B). In the samples with AAT and monomeric actin (left side of FIG. 10B), two higher molecular weight complexes were observed at 250 kDa and 180 kDa (lanes 2 and 3, 2:1 and 4:1 molar ratios, respectively). Identical bands were seen in 1 micron actin/AAT formulations (lane 10, 4:1 molar ratio). These particular molecular weight complexes were the same size as the bands seen in FIG. 10A which were probed with actin, suggesting that both actin and AAT were present in these complexes.

### Example 23

FIG. 11A depicts representative results from an experiment that tested AAT as an actin-binding protein. 4 μg of AAT was added to 40 μg of F-actin and incubated at room temperature for 30 min. Controls included incubations of AAT alone (left lane) or actin alone (middle lane). The samples were centrifuged at 150,000×g for 1 hr and the supernatants separated from the pellets and placed into separate tubes. Supernatants and pellets were denatured in SDS-PAGE gel loading buffer and electrophoresed into a 4-20% (w/v) SDS-PAGE gradient gel. They were stained with coomassie blue and the bands digitized and quantified using a densitometer. In the assay, proteins that bind F-actin are indicated by co-sedimentation with actin in the pellet. FIG. 11A shows a stained SDS-PAGE gel from a typical experiment. In the supernatant (upper panels), distinct bands for AAT (left lane) and actin (middle lane) was easily visualized and quantified. In the sample containing both AAT and actin (right lane), slightly less actin (approximately 5%) was recovered in the supernatant compared to the Actin only control (middle lane). In the pelletted samples (lower panels), AAT sedimented in both the control (AAT Only) and AAT+Actin samples, although more AAT pelletted with the addition of actin than in the AAT only control. Eight experimental replicates were performed to verify these results, and the data are shown in FIG. 11B. After setting the amount of AAT sedimented in the AAT only samples to 100%, the percentage of AAT sedimented in the presence of actin was calculated and found to be statistically significant (268%, or 1.7-fold increase in actin-sedimented AAT, P<0.001).

The observation that increased amounts of AAT sedimented in the presence of F-actin than in the AAT only control appear to indicate that AAT binds to F-actin.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed:

1. A method for treating a subject for actin-mediated medical condition, said method comprising administering to the subject a therapeutically effective amount of an actin modulator, whereby the medical condition of the subject is treated at least in part due to modulation of the cell-associated actin activity by the substance.

2. The method of claim 1, wherein the medical condition is an intracellular actin-mediated medical condition.

3. The method of claim 1, wherein the medical condition is selected from the group consisting of viral infection, bacterial infection, conditions mediated by pro-inflammatory cytokine production, a tumor, and graft rejection.

4. The method of claim 3, wherein the medical condition is mediated by bacteria-produced toxins.

5. The method of claim 3, wherein the medical condition is selected from the group consisting of lung transplant, liver transplant, heart transplant, kidney transplant, bone marrow transplant and pancreatic islet transplant.

6. The method of claim 3, wherein the medical condition is a retroviral infection.

7. The method of claim 6, wherein the medical condition is human immunodeficiency virus (HIV) infection.

8. The method of claim 3, wherein the medical condition is mycobacterial infection.

9. The method of claim 3, wherein the medical condition is selected from the group consisting of fibrosarcoma, myxosarcoma, liposarcoma, chondrosarcoma, osteogenic sarcoma, chordoma, angiosarcoma, endotheliosarcoma, lymphangiosarcoma, Kaposi's sarcoma, lymphangioendotheliosarcoma, synovioma, mesothelioma, Ewing's tumor, leiomyosarcoma, rhabdomyosarcoma, rhabdosarcoma, colorectal carcinoma, pancreatic cancer, breast cancer, ovarian cancer, prostate cancer, melanoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, myeloma, lymphoma, and leukemia.

10. The method of claim 3, wherein the medical condition is selected from the group consisting of lupus, rheumatoid arthritis, inflammatory bowel disease, sepsis, and sero-negative spondyloarthropathies.

11. The method of claim 1, wherein the medical condition is a disease mediated by apoptosis or NO production.

12. The method of claim 11, wherein the apoptosis mediated disease is selected from the group consisting of sepsis, bacterial meningitis, and ischemia-reperfusion injury.

13. The method of claim 12, wherein ischemia-reperfusion injury is selected from the group consisting of myocardial infarction, stroke, ischemic nephropathy, acute respiratory distress syndrome (ARDS), and shock liver.

14. The method of claim 1, wherein the actin modulator is an actin-binding molecule or an antagonist of actin polymerization.

15. The method of claim 1, wherein the actin modulator inhibits the polymerization of actin.

16. The method of claim 1, wherein the actin modulator is selected from the group consisting of Gelsolin, latrunculin, vitrunculin, cytochalasin D, anti-actin antibodies, or a derivative or an analog thereof.

17. The method of claim 1, wherein the actin modulator is α₁-antitrypsin (AAT) or a member of the SERPIN (serine protease inhibitor) family of natural proteins, or a derivative thereof.

18. The method of claim 17, wherein the actin modulator is α₁-antitrypsin (AAT) or a derivative thereof.

19. The method of claim 18, wherein the actin modulator is selected from the group consisting of FVFLM (SEQ ID NO:1), FVFAM (SEQ ID NO:2), FVALM (SEQ ID NO:3), FVFLA (SEQ ID NO:4), FLVFI (SEQ ID NO:5), FLMII (SEQ ID NO:6), FLFVL (SEQ ID NO:7), FLFVV (SEQ ID NO:8), FLFLI (SEQ ID NO:9), FLFFI (SEQ ID NO: 10), FLMFI (SEQ ID NO: 11), FMLLI (SEQ ID NO: 12), FIIMI (SEQ ID NO: 13), FLFCI (SEQ ID NO: 14), FLFAV (SEQ ID NO: 15), FVYLI (SEQ ID NO: 16), FAFLM (SEQ ID NO: 17), AVFLM (SEQ ID NO: 18), and a combination thereof.

20. A method for treating a subject for actin-mediated medical condition, said method comprising administering to the subject a therapeutically effective amount of an actin modulator that binds to cell-surface actin, whereby the medical condition of the subject is treated at least in part due to modulation of the cell-surface actin activity by the actin modulator.
